(19) United States
(12) Patent Application Publication
(10) Pub. No.: US 2025/0354120 A1
(43) Pub. Date: Nov. 20, 2025

(54) METHODS AND SYSTEMS FOR CO-DIFFERENTIATION OF CELLS USING OPTOGENETICS

(71) Applicant: Prolific Machines Inc., Emeryville, CA (US)

(72) Inventors: Deniz KENT, Oakland, CA (US); Maximiliaan HUISMAN, Oakland, CA (US); Declan JONES, Oakland, CA (US); Victor JONES, Berkeley, CA (US); Monique HOOLEY, Oakland, CA (US); Emily GALE, Berkeley, CA (US); Maximilian HÖRNER, Emeryville, CA (US)

(21) Appl. No.: 19/224,087

(22) Filed: May 30, 2025

Related U.S. Application Data
(63) Continuation of application No. PCT/US2023/082116, filed on Dec. 1, 2023.
(60) Provisional application No. 63/385,716, filed on Dec. 1, 2022.

(51) Int. Cl. C12N 5/077; C12N 5/10

(52) U.S. Cl. CPC C12N 5/0653; C12N 5/0655; C12N 5/0658; C12N 5/10; C12N 2501/00; C12N 2506/1315; C12N 2510/00; C12N 2529/10

(57) ABSTRACT

Provided herein are methods and systems for co-differentiating differentiatable cells in a population of differentiatable cells into two or more different cell lineages. In some embodiments, the methods and systems use light- or chemically-activatable recombinases to drive expression of one or more transcription factors and/or differentiation factors.

### CROSS-REFERENCE

This application is a continuation of International Application No. PCT/US2023/082116, filed Dec. 1, 2023, which claims the benefit of U.S. Provisional Application No. 63/385,716, filed on Dec. 1, 2022, each of which is incorporated herein by reference in its entirety.

### BACKGROUND

Transcription factors can regulate the differentiation of cells into different cell types. For example, expression of transcription factors such as MyoD can result in cells differentiating into muscle cells, while expression of other transcription factors such as PPARγ can cause cells to differentiate into adipocytes. However, co-differentiating cells from the same population of cells into multiple, different cell lineages with high temporal and spatial precision is not currently possible using existing technology.

### SUMMARY

There is an unmet need for methods and systems for co-differentiating cells within a population of cells into multiple cell lineages with high temporal and spatial precision. The methods and systems described herein meet this unmet need.

In one aspect, a method of co-differentiating a population of differentiatable cells is provided, the method comprising: (a) providing or obtaining a population of differentiatable cells; (b) controlling differentiation of a first differentiatable cell of the population of differentiatable cells with light, thereby differentiating the first differentiatable cell into a first cell lineage; and (c) differentiating a second differentiatable cell of the population of differentiatable cells into a second cell lineage, wherein the first cell lineage and the second cell lineage are different, thereby co-differentiating the population of differentiatable cells. In some cases, the controlling differentiation of (b) comprises illuminating the first differentiatable cell with light at a first wavelength or wavelength range. In some cases, the controlling differentiation of (b) comprises removing light at a first wavelength or wavelength range from the first differentiatable cell. In some cases, the differentiating of (c) comprises illuminating the second differentiatable cell of the population of differentiatable cells with light at a second wavelength or wavelength range to differentiate the second differentiatable cell into the second cell lineage; or the differentiating of (c) comprises removing light at a second wavelength or wavelength range from the second differentiatable cell to differentiate the second differentiatable cell into the second cell lineage. In some cases, the first wavelength or wavelength range and the second wavelength or wavelength range are different. In some cases, the differentiating of (c) comprises contacting the second differentiatable cell of the population of differentiatable cells with a chemical inducer to differentiate the second differentiatable cell into the second cell lineage; or the differentiating of (c) comprises removing a chemical inducer from the second differentiatable cell of the population of differentiatable cells to differentiate the second differentiatable cell into the second cell lineage. In some cases, each differentiatable cell of the population of differentiatable cells is engineered to contain an exogenous nucleic acid comprising: (i) a nucleic acid sequence encoding for at least one first transcription factor or first differentiation factor that effects differentiation into the first cell lineage; and (ii) a nucleic acid sequence encoding for at least one second transcription factor or second differentiation factor that effects differentiation into the second cell lineage. In some cases, the controlling of (b) comprises modulating expression of the at least one first transcription factor or first differentiation factor, and the differentiating of (c) comprises modulating expression of the at least one second transcription factor or second differentiation factor. In some cases, the exogenous nucleic acid comprises at least one promoter operably linked to the nucleic acid sequence encoding the at least one first transcription factor or first differentiation factor and nucleic acid sequence encoding the at least one second transcription factor or second differentiation factor. In some cases, the at least one promoter is a constitutive promoter. In some cases, the exogenous nucleic acid further comprises: (iii) a blocking sequence downstream of the at least one promoter which, when present, blocks expression of the at least one first transcription factor or first differentiation factor and/or the at least one second transcription factor or second differentiation factor. In some cases, the nucleic acid sequence encoding the at least one first transcription factor or first differentiation factor, or a portion thereof, is present in the exogenous nucleic acid in reverse orientation such that the at least one first transcription factor or first differentiation factor is not expressed. In some cases, the nucleic acid sequence encoding the at least one second transcription factor or second differentiation factor, or a portion thereof, is present in the exogenous nucleic acid in reverse orientation such that the at least one second transcription factor or second differentiation factor is not expressed. In some cases, each differentiatable cell of the population of differentiatable cells further comprises: (iv) an exogenous nucleic acid sequence encoding a first activatable recombinase; and (v) an exogenous nucleic acid sequence encoding a second activatable recombinase. In some cases, the first activatable recombinase is a light-activatable recombinase. In some cases, the second activatable recombinase is a light-activatable recombinase. In some cases, the first activatable recombinase and the second activatable recombinase are different. In some cases, the second activatable recombinase is a chemically-activatable recombinase. In some cases, expression of the first activatable recombinase, expression of the second activatable recombinase, or both, is induced by a chemical inducer, or is induced by light. In some cases, the blocking sequence is flanked by a first recombinase recognition site that is recognized by the first activatable recombinase, a second recombinase recognition site that is recognized by the second activatable recombinase, or both. In some cases, the controlling of (b), the differentiating of (c), or both, results in excision of the blocking sequence thereby inducing expression of the at least one first transcription factor or first differentiation factor, the at least one second transcription factor or second differentiation factor, or both. In some cases, (i) the nucleic acid sequence encoding the at least one first transcription factor or first differentiation factor, or the portion thereof, present in the exogenous nucleic acid in reverse orientation; (ii) the nucleic acid sequence encoding the at least one second transcription factor or second differentiation factor, or the portion thereof, present in the exogenous nucleic acid sequence in reverse orientation; or (iii) both, is flanked by a first recombinase recognition site that is recognized by the first activatable recombinase, a second recombinase recognition site that is recognized by the second activatable recombinase, or both. In some cases, the controlling of (b), the differentiating of (c), or both, results in (i) inversion of the nucleic acid sequence encoding the at least one first transcription factor or first differentiation factor, or the portion thereof, such that the at least one first transcription factor or first differentiation factor is expressed; (ii) inversion of the nucleic acid sequence encoding the at least one second transcription factor or second differentiation factor, or the portion thereof, such that the at least one second transcription factor or second differentiation factor is expressed; or (iii) both. In some cases, the method further comprises (d) differentiating a third differentiatable cell of the population of differentiatable cells into a third cell lineage, wherein the third cell lineage is different from the first cell lineage and the second cell lineage. In some cases, the differentiating of (d) comprises (i) illuminating the third differentiatable cell with light at a third wavelength or wavelength range; (ii) removing light at a third wavelength or wavelength range from the third differentiatable cell; (ii) contacting the third differentiatable cell with a chemical inducer; or (iv) removing a chemical inducer from the third differentiatable cell. In some cases, the third wavelength or wavelength range is different from the first wavelength or wavelength range, the second wavelength or wavelength range, or both. In some cases, the population of differentiatable cells comprises stem cells. In some cases, the stem cells are pluripotent stem cells or multipotent stem cells. In some cases, the population of differentiatable cells comprise mature somatic cells capable of transdifferentiation under certain conditions. In some cases, the differentiatable cells are human differentiatable cells or bovine differentiatable cells. In some cases, the first cell lineage, the second cell lineage, and/or the third cell lineage are selected from the group consisting of: an adipocyte, a myocyte, and a chondrocyte. In some cases, the at least one first transcription factor, the at least one second transcription factor, or both, is selected from the group consisting of: PPARγ, CEBP alpha, MYOD, MYOG, MYF5, MRF4 (MYF6), HEYL, KLF4, PAX3, PRDM16, SREBP1, SOX9, SOX5, SOX6, and any combination thereof. In some cases, the controlling of (b) and the differentiating of (c) occur substantially simultaneously. In some cases, the controlling of (b) and the differentiating of (c) occur sequentially. In some cases, the controlling of (b) precedes the differentiating of (c), or the differentiating of (c) precedes the controlling of (b). In some cases, the first differentiatable cell and the second differentiatable cell are adjacent to each other. In some cases, the controlling of (b) further comprises illuminating a plurality of first differentiatable cells with light at the first wavelength or wavelength range to differentiate each of the plurality of first differentiatable cells into the first cell lineage; or the controlling of (b) further comprises removing light at the first wavelength or wavelength range from the plurality of first differentiatable cells to differentiate each of the plurality of first differentiatable cells into the first cell lineage. In some cases, the differentiating of (c) further comprises differentiating a plurality of second differentiatable cells to differentiate each of the plurality of second differentiatable cells into the second cell lineage. In some cases, the population of differentiatable cells are deposited onto a solid support. In some cases, the population of differentiatable cells are deposited on the solid support in multiple layers. In some cases, the solid support is coated with one or more extracellular matrix components, or portions thereof.

In another aspect, a system for co-differentiating a population of differentiatable cells is provided, the system comprising: (a) a population of differentiatable cells, wherein each differentiatable cell of the population of differentiatable cells is engineered to contain an exogenous nucleic acid comprising: (i) a nucleic acid sequence encoding for at least one first transcription factor or first differentiation factor that effects differentiation into a first cell lineage; and (ii) a nucleic acid sequence encoding for at least one second transcription factor or second differentiation factor that effects differentiation into a second cell lineage; and (b) one or more light source configured to control differentiation of a first differentiatable cell of the population of differentiatable cells with light at a first wavelength or wavelength range to differentiate the first differentiatable cell into the first cell lineage, wherein the first cell lineage and the second cell lineage are different. In some cases, the system is configured to illuminate the first differentiatable cell with light at the first wavelength or wavelength range using the one or more light source to differentiate the first differentiatable cell into the first cell lineage. In some cases, the system is configured to remove light at the first wavelength or wavelength range from the first differentiatable cell to differentiate the first differentiatable cell into the first cell lineage. In some cases, the system is further configured to illuminate a second differentiatable cell of the population of differentiatable cells with light at a second wavelength or wavelength range using the one or more light source to differentiate the second differentiatable cell into the second cell lineage; or the system is further configured to remove light at the second wavelength or wavelength range from the second differentiatable cell to differentiate the second differentiatable cell into the second cell lineage. In some cases, the first wavelength or wavelength range and the second wavelength or wavelength range are different. In some cases, the system further comprises a chemical inducer to differentiate a second differentiatable cell of the population of differentiatable cells into the second cell lineage; or the system is further configured to remove a chemical inducer from a second differentiatable cell to differentiate the second differentiatable cell into the second cell lineage. In some cases, the exogenous nucleic acid comprises at least one promoter operably linked to the at least one first transcription factor or first differentiation factor and the at least one second transcription factor or second differentiation factor. In some cases, the at least one promoter is a constitutive promoter. In some cases, the exogenous nucleic acid further comprises: (iii) a blocking sequence downstream of the at least one promoter which, when present, blocks expression of the at least one first transcription factor or first differentiation factor and the at least one second transcription factor or second differentiation factor. In some cases, the nucleic acid sequence encoding the at least one first transcription factor or first differentiation factor, or a portion thereof, is present in the exogenous nucleic acid in reverse orientation such that the at least one first transcription factor or first differentiation factor is not expressed. In some cases, the nucleic acid sequence encoding the at least one second transcription factor or second differentiation factor, or a portion thereof, is present in the exogenous nucleic acid in reverse orientation such that the at least one second transcription factor or second differentiation factor is not expressed. In some cases, each differentiatable cell of the population of differentiatable cells further comprises: (iv) a nucleic acid sequence encoding a first activatable recombinase; and (v) a nucleic acid sequence encoding a second activatable recombinase. In some cases, the first activatable recombinase is a light-activatable recombinase. In some cases, the second activatable recombinase is a light-activatable recombinase. In some cases, the first activatable recombinase and the second activatable recombinase are different. In some cases, the second activatable recombinase is a chemically-activatable recombinase. In some cases, expression of the first activatable recombinase, expression of the second activatable recombinase, or both, is inducible by a chemical inducer or is inducible by light. In some cases, the blocking sequence is flanked by a first recombinase recognition site that is recognized by the first activatable recombinase, a second recombinase recognition site that is recognized by the second activatable recombinase, or both. In some cases, the blocking sequence is configured to be excised by the first activatable recombinase, the second activatable recombinase, or both, thereby modulating expression of the at least one first transcription factor or first differentiation factor, the at least one second transcription factor or first differentiation factor, or both. In some cases, (i) the nucleic acid sequence encoding the at least one first transcription factor or first differentiation factor, or the portion thereof, present in the exogenous nucleic acid in reverse orientation; (ii) the nucleic acid sequence encoding the at least one second transcription factor or second differentiation factor, or the portion thereof, present in the exogenous nucleic acid sequence in reverse orientation; or (iii) both, is flanked by a first recombinase recognition site that is recognized by the first activatable recombinase, a second recombinase recognition site that is recognized by the second activatable recombinase, or both. In some cases, (i) the nucleic acid sequence encoding the at least one first transcription factor or first differentiation factor, or the portion thereof, is configured to be inverted by the first activatable recombinase, the second activatable recombinase, or both, such that the at least one first transcription factor or first differentiation factor is expressed; (ii) the nucleic acid sequence encoding the at least one second transcription factor or second differentiation factor, or the portion thereof, is configured to be inverted by the first activatable recombinase, the second activatable recombinase, or both, such that the at least one second transcription factor or second differentiation factor is expressed; or (iii) both. In some cases, the exogenous nucleic acid further comprises a nucleic acid sequence encoding for at least one third transcription factor or third differentiation factor that effects differentiation into a third cell lineage. In some cases, the system is further configured to illuminate a third differentiatable cell of the population of differentiatable cells with light at a third wavelength or wavelength range using the one or more light source to differentiate the third differentiatable cell into the third cell lineage; or wherein the system is further configured to remove light at the third wavelength or wavelength range to differentiate the third differentiatable cell into the third cell lineage, wherein the third wavelength or wavelength range is different from the first wavelength or wavelength range and/or the second wavelength or wavelength range, and wherein the third cell lineage is different from the first cell lineage and/or the second cell lineage. In some cases, the differentiatable cells are stem cells. In some cases, the stem cells are pluripotent stem cells or multipotent stem cells. In some cases, the differentiatable cells are mature, somatic cells capable of being transdifferentiated under certain conditions. In some cases, the differentiatable cells are human differentiatable cells or bovine differentiatable cells. In some cases, the first cell lineage, the second cell lineage, and/or the third cell lineage is selected from the group consisting of: an adipocyte, a myocyte, and a chondrocyte. In some cases, the at least one first transcription factor, the at least one second transcription factor, the at least one third transcription factor, or any combination thereof, is selected from the group consisting of: PPARγ, CEBP alpha, MYOD, MYOG, MYF5, MRF4 (MYF6), HEYL, KLF4, PAX3, PRDM16, SREBP1, SOX9, SOX5, SOX6, and any combination thereof. In some cases, the first differentiatable cell and the second differentiatable cell are adjacent to each other. In some cases, the system further comprises a solid support, wherein the population of differentiatable cells are deposited onto the solid support. In some cases, the population of differentiatable cells are deposited on the solid support in multiple layers. In some cases, the one or more light comprises one or more light-emitting diodes (LEDs). In some cases, the one or more LEDs comprises at least two different LEDs. In some cases, the at least two different LEDs emit light at different wavelengths or wavelength ranges. In some cases, the one or more light source comprises one or more lasers. In some cases, the one or more light source comprises an incandescent light source.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a non-limiting example of a schematic of a nucleic acid sequence for expression of a transcription factor that is suitable for use with the methods and systems described herein.

FIG. 2 depicts a non-limiting example of an inducible recombinase system that is suitable for use with the methods and systems described herein.

FIG. 3 describes a non-limiting example of using a recombinase to invert a nucleic acid sequence encoding transcription factor(s) or differentiation factor(s) such that the desired transcription factor(s) or differentiation factor(s) are expressed.

FIG. 4 schematically illustrates a non-limiting example of co-differentiation of a population of differentiatable cells into multiple cell lineages using the methods and systems described herein.

FIGS. 5A-5C schematically illustrate a non-limiting example of constructs and co-differentiation mechanism described herein.

FIGS. 6A and 6B depict spatial and temporal optogenetic control of expression of green fluorescent protein (GFP).

FIGS. 7A-7C demonstrate changes in gene expression in response to different light parameters: blue light (FIG. 7A), UV light (FIG. 7B), and red light (FIG. 7C).

FIG. 8 demonstrates co-differentiation of a population of differentiatable cells using blue light and doxycycline.

FIG. 9 demonstrates co-differentiation of a population of differentiatable cells using UV light and doxycycline.

FIGS. 10A and 10B show quantitative data of fat differentiation via blue light system (FIG. 10A) and UV light system (FIG. 10B).

FIG. 11 shows transcription factors or differentiation factors that are capable of inducing muscle differentiation.

### DETAILED DESCRIPTION

The developmental fate of a differentiatable cell can be determined by expressing particular transcription factors or differentiation factors in the cell that set in motion a developmental program leading to its differentiation into a particular cell type. However, differentiating a population of differentiatable cells into multiple cell types in a (e.g., temporally, spatially) controlled manner (e.g. co-differentiation) is not currently possible using existing technology. For instance, patterning differentiatable cells into a three-dimensional tissue requires differentiation of differentiatable cells into multiple cell types with high spatiotemporal precision. Controlling the temporal and spatial expression of transcription factors or differentiation factors in differentiatable cells allows for (e.g., simultaneous) co-differentiation of the differentiatable cells into multiple cell types within a single population. The methods and systems disclosed herein generally use at least one light-activatable recombinase to differentiate a first cell into a first cell lineage. The second cell may be differentiated into a second cell lineage by any mechanism, including, as described herein, by use of a second light-activatable recombinase, by expressing one or more transcription factors and/or differentiation factors using an inducible promoter (e.g., inducible by light, inducible by chemical), by contacting the cell with appropriate culture media factors, by cell-cell contact mediated differentiation, among others.

In some embodiments, the methods and systems provided herein use optogenetics for (e.g., spatially, temporally) controlling co-differentiation of cells into multiple cell lineages. For instance, in some embodiments, a promoter regulating a transcription factor may be induced (e.g., a light-inducible promoter) by illumination with light at a particular wavelength or wavelength range. In some embodiments, the methods and systems provided herein use recombinases. A recombinase recognizes a specific DNA sequence, and if there are two recognition sequences in the proper arrangement, it can excise or invert the orientation of the DNA between the two sites. By briefly activating the recombinase, a permanent change can be made to the DNA, which offers the prospect of permanently switching on the differentiation genes with only a short activation phase.

Disclosed herein are methods and systems for co-differentiating differentiatable cells within a population of differentiatable cells into multiple cell lineages. The methods and systems, in some cases, include the use of recombinases, such as recombinases that can be activated by light, and/or by chemical means.

### Methods

Provided herein are methods for co-differentiating a population of differentiatable cells. The methods may comprise providing or obtaining a population of differentiatable cells (e.g., stem cells), controlling differentiation of a first differentiatable cell of the population of differentiatable cells with light to differentiate the first differentiatable cell into a first cell lineage, and differentiating a second differentiatable cell into a second cell lineage. In some cases, the first differentiatable cell lineage and the second differentiatable cell lineage are different, such that the methods allow for co-differentiating the first differentiatable cell and the second differentiatable cell into different cell lineages within the same population of differentiatable cells. In some instances, controlling differentiation of the first differentiatable cell with light involves illuminating the first differentiatable cell with light at a first wavelength or wavelength range to differentiate the first differentiatable cell into the first cell lineage. In other instances, controlling differentiation of the first differentiatable cell with light involves removing light at a first wavelength or wavelength range from the first differentiatable cell. For example, a light source may be turned off such that the first differentiatable cell is no longer illuminated with light at any wavelength. In other instances, a light may be changed from the first wavelength or wavelength range to a different wavelength or wavelength range, such that the cell is no longer illuminated with light at the first wavelength or wavelength range.

In some instances, the methods described herein involve co-differentiation of cells within a population of differentiatable cells into multiple cell lineages. In some instances, the methods involve co-differentiation of differentiatable cells within a population of differentiatable cells into at least two, at least three, at least four, or at least five different cell lineages.

Expression of a transcription factor and/or differentiation factor or combination of transcription factors and/or differentiation factors in a differentiatable cell may result in differentiation of the differentiatable cell into a particular cell type. Any transcription factor and/or differentiation factor or combination of transcription factors and/or differentiation factors that, when expressed, leads to differentiation of a differentiatable cell to a desired cell lineage is contemplated herein. In a non-limiting example, when differentiation of a differentiatable cell into a fat cell (e.g., adipocyte) is desired, the differentiatable cell may be induced to express a transcription factor or differentiation factor such as, but not limited to, SREBP1, PPARγ, and/or CEBP alpha. In another non-limiting example, when differentiation of a differentiatable cell into a muscle cell (e.g., myocyte) is desired, the differentiatable cell may be induced to express a transcription factor or differentiation factor such as, but not limited to, MYOD, MYOG, MYF5, MRF4 (MYF6), HEYL, KLF4, and/or PAX3. In another non-limiting example, when differentiation of a differentiatable cell into a cartilage cell (e.g., chondrocyte) is desired, the differentiatable cell may be induced to express a transcription factor or differentiation factor such as, but not limited to, SOX9, SOX5, and/or SOX6. In some embodiments, the at least one transcription factor is selected from the group consisting of PPARγ, CEBP alpha, MYOD, MYOG, and a combination thereof. In some cases, expression of the transcription factor and/or differentiation factor may be regulated e.g., by a chemical inducer or by light.

It should be understood that the disclosure is not limited to expression of transcription factors. Any differentiation factor necessary or sufficient, either alone or in combination with any other factor, may be used in the methods and systems provided herein to co-differentiate a population of differentiatable cells. For example, non-transcription factors may be used, such as chromatin remodeling factors. In some cases, the chromatin remodeling factor may be SMARCD3 and/or JMJD3.

In certain aspects, the methods described herein may involve expressing at least one, at least two, at least three, at least four, at least five, at least six, at least seven, at least eight, at least nine, or at least ten transcription factors and/or other differentiation factors (e.g., chromatin remodeling factors) in the differentiatable cell (e.g., to differentiate the differentiatable cell into a desired cell lineage).

In some aspects, the differentiatable cells described herein comprise at least one exogenous nucleic acid. The exogenous nucleic acid may comprise a sequence encoding any transcription factor and/or other differentiation factor (e.g., chromatin remodeling factors) as described herein. The sequence encoding the transcription factor and/or other differentiation factor may be operably linked to a promoter. The nucleic acids described herein may comprise promoter sequences. The promoter sequences may be constitutively active. In an alternate embodiment, a promoter sequence may be conditionally active. For instance, a promoter sequence may be inducible, e.g., by a chemical inducer or by light.

In some aspects, the differentiatable cells described herein comprise at least two exogenous nucleic acids. In some aspects, the differentiatable cells described herein comprise at least three, at least four, or at least five exogenous nucleic acids. Each exogenous nucleic acid may comprise at least one nucleic acid sequence encoding a transcription factor and/or a differentiation factor. The nucleic acid sequence encoding the transcription factor and/or a differentiation factor may be operably linked to a promoter, as described herein. In some cases, when more than one transcription factor and/or differentiation factor is used to differentiate a differentiatable cell to a desired cell lineage, expression of each of the more than one transcription factor and/or differentiation factor may be under the control of the same, single promoter. In such cases, each gene encoding the transcription factor and/or differentiation factor may be combined into a single transcript. The single transcript may either encode self-cleaving 2A peptides between each separate protein, or may contain internal ribosome entry sites.

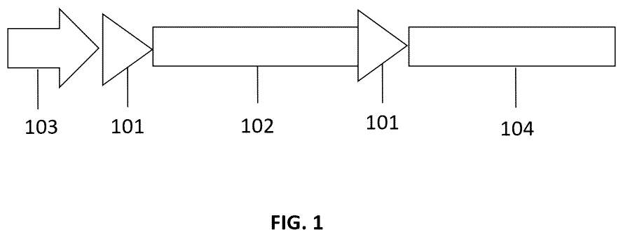

In a non-limiting example, the exogenous nucleic acid may be arranged as depicted in FIG. 1. A promoter 103 may be operably linked to a nucleic acid sequence encoding a transcription factor or differentiation factor 104. In some cases, a blocking sequence 102 may be located between the promoter sequence and the nucleic acid sequence encoding the transcription factor or differentiation factor. A blocking sequence may be any nucleic acid sequence that prevents transcription of the nucleic acid sequence encoding the transcription factor, such as a nucleic acid sequence encoding one or more stop codons and/or a transcription terminator sequence. In some cases, the blocking sequence may comprise an expression cassette or multiple expression cassettes, each containing a transcription terminator sequence. In some cases, the blocking sequence may be flanked by recombinase recognition sites 101. In such scenarios, in the presence of a recombinase, the blocking sequence is excised allowing for expression of the transcription factor or differentiation factor. When a target sequence (e.g., a blocking sequence) is “flanked” by recombinase recognition sites, what is meant is that there is at least a first recombinase recognition site upstream (e.g., 5′) of the target sequence, and at least a second recombinase recognition site downstream (e.g., 3′) of the target sequence. The term “flanked” when used in relation to a recombinase recognition site includes scenarios in which the recombinase recognition site(s) is directly adjacent to or directly connected to the target sequence, as well as scenarios in which there is intervening sequence of any length between the recombinase recognition site and the target sequence.

In some cases, the recombinase may be activatable, e.g., by a chemical activator, or by light, as described herein, thus allowing for control of recombinase activity. In such cases, the recombinase may be expressed in the differentiatable cell but may be in an inactive state until the differentiatable cell is exposed to the activator (e.g., chemical activator, light at a particular wavelength or wavelength range). Upon exposure of the differentiatable cell to the activator (e.g., chemical activator, light at a particular wavelength or wavelength range), the recombinase may be activated, leading to excision of the blocking sequence and expression of the transcription factor or differentiation factor (thereby leading to differentiation of the differentiatable cell into a desired cell lineage). In some cases, the recombinase is a light-activatable recombinase (e.g., comprises a light-activatable domain) that is activated by light at a particular wavelength or wavelength range. In some cases, the light-activatable recombinase may be deactivated by light at a wavelength or wavelength range that is different from the wavelength or wavelength range used to activate the recombinase. In some cases, the recombinase is a chemically-activatable recombinase.

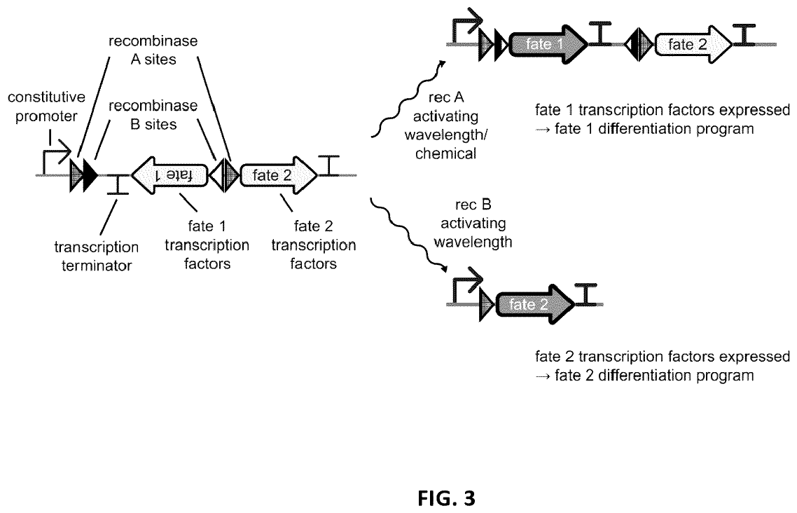

In an alternative embodiment, rather than excision of a blocking sequence, the activatable recombinase may be used to invert nucleic acid sequences such that the nucleic acid sequences are under the control of a promoter, thereby resulting in expression of the transcription factor and/or differentiation factor. FIG. 3 depicts a non-limiting example of this scenario. In this scenario, the nucleic acid sequence encoding one or more transcription factors and/or differentiation factors that, when expressed in a cell, effect differentiation into a muscle cell lineage are present in the exogenous nucleic acid in the reverse orientation (such that the one or more transcription factors and/or differentiation factors are not expressed). The muscle cell lineage cassette also serves as a blocking sequence such that the downstream fat cell lineage transcription factors and/or differentiation factors are not expressed. The nucleic acid sequence encoding the muscle cell lineage transcription factors and/or differentiation factors are flanked by two sets of recombinase recognition sites (e.g., recA, recB). Upon activation of an activatable recombinase (e.g., by light, by chemical) that recognizes recA sites, the recA-recognizing recombinase inverts the nucleic acid sequence encoding the muscle cell lineage transcription factors and/or differentiation factors such that the nucleic acid sequence is in the correct orientation and expression of the muscle cell lineage transcription factors and/or differentiation factors occurs. Expression of the muscle cell lineage transcription factors and/or differentiation factors causes differentiation of this cell into a muscle cell. Alternatively, when fat is desired, a second, different activatable recombinase is activated (e.g., by light) which recognizes recB sites. Upon activation of the recB-recognizing recombinase, the muscle cell lineage cassette is excised and the fat cell lineage transcription factors and/or differentiation factors are expressed. Expression of the fat cell lineage transcription factors and/or differentiation factors causes differentiation of this cell into a fat cell.

In some cases, more than one recombinase may be used in a population of differentiatable cells, such that, depending upon which recombinase is activated, a different transcriptional program is activated, such as the example described in FIG. 3. For example, a differentiatable cell may express a light-activatable recombinase (recombinase A) and a chemically-activatable recombinase (recombinase B). When differentiation into cell type A (e.g., muscle cells) is desired, the differentiatable cell may be exposed to the chemical activator, thereby activating recombinase A resulting in expression of transcription factor A and differentiation of the differentiatable cell into cell type A. When differentiation into cell type B (e.g., fat cells) is desired, the differentiatable cell may be exposed to light at a particular wavelength or wavelength range, thereby activating recombinase B resulting in expression of transcription factor B and differentiation of the differentiatable cell into cell type B. In a population of differentiatable cells, this method may be employed to temporally and/or spatially control co-differentiation of differentiatable cells into different cell lineages. In some cases, this method may be employed to simultaneously co-differentiate differentiatable cells into different cell lineages.

In some cases, the differentiatable cell may express two recombinases, each activatable by light at different wavelengths or wavelength ranges. In an alternative embodiment, the differentiatable cell may express two recombinases, one activatable by light at a particular wavelength or wavelength range, and the other by a chemical activator. In another embodiment, the differentiatable cell may express three recombinases, two activatable by light at different wavelengths or wavelength ranges, and the third activatable by a chemical activator. In some cases, the recombinases may be activated substantially simultaneously or simultaneously. In other cases, the recombinases may be activated sequentially. In some scenarios, only one recombinase is used to effect differentiation into a first cell lineage. The second, different cell lineage may be achieved by any other mechanism, such as by controlling expression of transcription factors and/or differentiation factors using an inducible promoter (e.g., inducible by chemical, inducible by light), by exposing the cells to a specific culture media or culture media factors, or by cell-cell contact mediated differentiation.

In some instances, the recombinase may be activated using an activatable system. The activatable system may be as described in Table 1.

TABLE 1
Activatable systems

| Purpose | Inducer | Elements |
|---|---|---|
| Chemically- | Abscisic acid | ABI & PYL1 fused to halves |
| activatable | | of a split recombinase |
| recombinase | | |
| Chemically- | Gibberellin | GID1 & GAI fused to halves |
| activatable | | of a split recombinase |
| recombinase | | |
| Optogenetically- | Light, wavelength | Split recombinase fused to |
| activatable | depends on which | halves of dimerizing opto pair |
| recombinase | optoswitches are | |
| | fused to the split | |
| | recombinase | |
| Optogenetically- | Blue light (460 nm) | LiCre |
| activatable | | |
| recombinase | | |
| Optogenetically- | Violet light (400 nm) | PhoCl, in the form |
| activatable | | SR-PhoCl-recombinase- |
| recombinase | | PhoCl-SR |

In various aspects, a combination of light-activatable domains (e.g., a first light-activatable domain and a second light-activatable domain) may be used (e.g., each of the light-activatable domains may be fused to a portion of the recombinase). In this scenario, the first light-activatable domain and the second light-activatable domain are binding partners, such that upon illumination with light at a particular wavelength or within a particular spectral range, the first and second light-activatable domains heterodimerize or hetero-oligomerize. The first and second light-activatable domains, upon illumination with light at a particular wavelength or within a particular spectral range, heterodimerize or hetero-oligomerize, thereby bringing the protein domains (or functional domains or functional portions thereof) into close contact with one another such that the recombinase(s) is/are activated. In some instances, the first and second light-activatable domains may be dissociated from one another (e.g., thereby deactivating the recombinase) upon illumination with light at a wavelength or wavelength range that is different from the wavelength or wavelength range used to heterodimerize or hetero-oligomerize the first and second light-activatable domains.

In various aspects, the light activatable domain comprises a Light-Oxygen-Voltage (LOV) photoreceptor domain, a LOV2 photoreceptor domain, a Cryptochrome (CRY) domain, Blue-light-using FAD (BLUF) photoreceptor domain, a Phytochrome (PHY) domain, CIB1 (cryptochrome-interacting basic-helix-loop-helix protein 1) (or a functional portion or domain thereof; e.g., CIBN (N-terminal domain of CIB1)), a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof.

In some instances, a combination of light-activatable domains is used, wherein the first light-activatable domain is cryptochrome 2 (or a variant or a functional portion thereof) and the second-light activatable domain is CIB1 (or a variant or a functional portion thereof; e.g., CIBN). In some instances, a combination of light-activatable domains is used, wherein the first light-activatable domain is BphP1 (or a variant or a functional portion thereof) and the second-light activatable domain is QPAS1 (or a variant or a functional portion thereof). In some cases, the light-activatable domain (or combination of light-activatable domains) is selected from Table 2. In some cases, the light-activatable domain may have an amino acid sequence having at least about 50% sequence identity (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater) to any one of the light-activatable domains described in Table 2.

TABLE 2
Non-limiting examples of light-activatable domain systems

| | | Binding | Binding | |
|---|---|---|---|---|
| System | Binding type | partner 1 | partner 2 | Light |
| Light-Oxygen- | Homodimer | VfAU1-LOV | — | Blue light results in |
| Voltage (LOV) | | | | dimerization |
| Cryptochrome | Homodimer/ | Photolyase- | — | Blue light results in |
| (CRY) | oligomer | Homologous | | dimerization |
| | | Region (PHR) | | |
| | | domain | | |
| CRY-CIBN | Heterodimer | PHR domain | CIBN | Blue light results in |
| | | | | dimerization |
| Blue-light- | Homodimer | BLUF domain | — | Blue light results in |
| using FAD | | | | dimerization |
| (BLUF) | | | | |
| photoreceptor | | | | |
| domain | | | | |
| PhyB/PIF | Heterodimer | PhyB | PIF (PIF3, | Red light results in |
| | | | PIF6) | dimerization, far red |
| | | | | light results in |
| | | | | dissociation |
| Dronpa | Homodimer | Dronpa | — | Illumination with blue |
| | | | | light results in |
| | | | | monomerization; |
| | | | | illumination with UV |
| | | | | light results in |
| | | | | dimerization |
| UVR8 | Homodimer | UVR8 | — | Dissociates when |
| | | | | exposed to UV-B light |
| UVR8 | Heterodimer | UVR8 | COP1 | UVR8 homodimer |
| | | | | dissociates when |
| | | | | exposed to UV-B light |
| | | | | and forms heterodimer |
| | | | | with COP1 |
| BphP1/QPAS1 | Heterodimer | BphP1 | QPAS-1 | Near infrared light |
| | | | | results in dimerization; |
| | | | | red light dissociates |
| Cobalamin | Oligomer | CBD | — | Homooligomerizes in |
| binding | | | | dark; green light results |
| domains | | | | in dissociation |
| (CBD) | | | | |
| MagRed | Heterodimer | DrBphP | Aff6_V18F | Red light results in |
| | | | ΔN | dimerization; near |
| | | | | infrared light |
| | | | | dissociates |
| PhyA/far-red | heterodimer | PhyA | FHY1, FHL | 660 nm light results in |
| elongated | | | or PIF | dimerization, 730 nm |
| hypocotyl 1 | | | | results in dissociation |
| (FHY1) or | | | | |
| FHY1-LIKE | | | | |
| (FHL) or PIF | | | | |
| Vivid (VVD) | Homodimer | VVD | — | Blue light results in |
| | | | | dimerization |

In another aspect of the methods described herein, the recombinase may be activated by a chemical inducer or by a specific wavelength or wavelength range of light. The recombinase may be constitutively expressed in an inactive form. The recombinase may be conditionally expressed by a chemically-induced or light-induced system (e.g., by use of an inducible (e.g., by chemical, by light) promoter) in an inactive form. The recombinase may mediate irreversible excision or may invert a nucleic acid sequence. The recombinase may be a serine integrase, such as ΦC31, TP901, and Bxb1. The recombinase may be a tyrosine recombinase, such as Cre, VCre and Flp.

In one embodiment, the recombinase is split into a first part and a second part. Addition of a chemical inducer results in dimerization and activation of the recombinase. The chemical inducer may be any of the chemical inducers described herein, including without limitations, rapamycin or derivatives thereof, ABA, GA, tetracycline or derivatives thereof, cumate or derivates thereof, or vanillic acid or derivatives thereof. The recombinase may be any recombinase, including, without limitations, Cre, VCre, Flp, ΦC31, TP901, and Bxb1.

In some embodiments, a chemical and/or light inducer controls expression of the recombinase and a chemical and/or light activator controls activation of the recombinase. In some instances, the chemical inducer and activator are both plant hormones. For instance, expression of the split recombinase may be regulated by GA while dimerization and activation of the split recombinase is mediated by ABA, or vice versa.

In another embodiment, dimerization and activation of the recombinase is induced by light (e.g., optogenetic dimerization). The first half of the recombinase and the second half of the recombinase may be fused to a light activatable domain. In various aspects, the light activatable domain comprises a Light-Oxygen-Voltage (LOV) photoreceptor domain, a LOV2 photoreceptor domain, a Cryptochrome (CRY) domain, Blue-light-using FAD (BLUF) photoreceptor domain, a Phytochrome (PHY) domain, CIB1 (cryptochrome-interacting basic-helix-loop-helix protein 1) (or a functional portion or domain thereof; e.g., CIBN), a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof. Regulation of the dimerization and activation of the recombinase may utilize any of the light activatable systems described in Table 2. Dimerization and activation may alternatively be induced by temperature. In some instances, deactivation of the recombinase may be achieved by illuminating the cells with light at a wavelength or wavelength range that is different from the wavelength or wavelength range used to activate the recombinase.

In various aspects, the methods involve exposing the differentiatable cells (e.g., genetically engineered to express the fusion protein comprising the recombinase and the light-activatable domain) with light at a particular wavelength or light within a particular spectral range. The wavelength or wavelength range of light is selected such that the light is capable of activating the light-activatable domain. For example, Table 2 provides non-limiting examples of light parameters for different light-activatable domain systems. The wavelength or wavelength range of light may be one or more of infrared, near infrared, visible light (e.g., red, green, blue), ultraviolet light, or a combination thereof. Infrared light may comprise light at a wavelength or wavelength range of about 780 nm to 1 mm. Near infrared light may comprise light at a wavelength or wavelength range of about 740 nm to about 780 nm. Red light may comprise light at a wavelength or wavelength range of about 620 nm to 750 nm, 600 nm to 690 nm, or about 650 nm. Green light may comprise light at a wavelength or wavelength range of about 577 nm to about 492 nm. Blue light may comprise light at a wavelength or wavelength range of 492 to about 455 nm, or about 440 nm to about 473 nm. Ultraviolet light may comprise light at a wavelength or wavelength range from about 10 nm to 400 nm, or from about 280 to 315 nm. In various aspects, the wavelength or wavelength range of light is from 100 nm to 1 mm.

In various aspects, the methods involve illuminating the cells with light having one or more illumination parameters. In some cases, the one or more illumination parameters includes light intensity and/or a temporal pattern of illumination. In some cases, the illumination intensities can be about 0 μW/mm² to about 100 μW/mm². In some cases, the illumination intensities can be at least or up to about 0 μW/mm², 0.1 μW/mm², 0.2 μW/mm², 0.3 μW/mm², 0.4 μW/mm², 0.5 μW/mm², 0.6 μW/mm², 0.7 μW/mm², 0.8 μW/mm², 0.9 μW/mm², 1 W/mm², 1.2 μW/mm², 1.4 W/mm², 1.6 μW/mm², 1.8 μW/mm², about 2 μW/mm², about 3 μW/mm², about 4 μW/mm², about 5 μW/mm², about 6 μW/mm², about 8 μW/mm², about 10 μW/mm², about 20 μW/mm², about 30 μW/mm², about 40 μW/mm², about 50 μW/mm², about 60 μW/mm², about 70 μW/mm², about 80 μW/mm², about 90 μW/mm², or about 100 μW/mm².

In some cases, the temporal pattern may include a stimulus duration and an interstimulus duration. In some cases, the temporal pattern comprises a light stimulus duration of at least about one tenth of a second, at least about 1 second, at least about 1 minute, at least about 5 minutes, at least about 30 minutes, or at least about 1 hour. In some cases, the stimulus duration may be at least about 5 minutes. In some cases, the temporal pattern comprises an interstimulus duration of at least about 1 second, at least about 1 minute, at least about 5 minutes, at least about 10 minutes, at least about 30 minutes, at least about 1 hour, at least about 2 hours, at least about 3 hours, at least about 4 hours, at least about 5 hours, or greater. In some cases, the interstimulus duration may be from about 20 minutes to about 250 minutes.

In another embodiment, the recombinase comprises a single chain polypeptide. The single chain polypeptide may be fused to a light activatable domain to create a light-activatable recombinase. Exposure to light at a particular wavelength or wavelength range may result in activity of the recombinase. For instance, illumination of the AsLOV2-based Cre system LiCre with blue light results in activation of the recombinase.

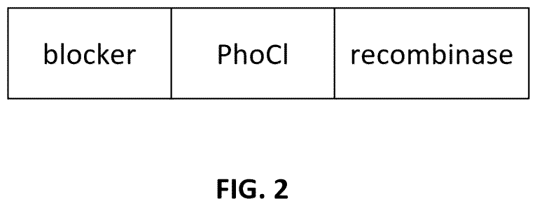

In another embodiment, the recombinase may be fused to a PhoCl protein or a derivative thereof. Illumination with violet light (about 400 nm) results in cleavage of the PhoCl. In some instances, a PhoCl domain may be present in the fusion protein between a blocker domain and the recombinase domain, as depicted in FIG. 2. The blocker domain may be any domain that prevents the recombinase from functioning, such as a domain that prevents the recombinase from entering the nucleus. For instance, the blocker domain may be a steroid receptor domain which interacts with Hsp90 to prevent nuclear entry of the recombinase. Exposing the fusion protein comprising the blocker domain, the PhoCl domain, and the recombinase to violet light may result in cleavage of the PhoCl domain, allowing the recombinase to enter the nucleus and reach the genomic DNA, resulting in recombinase activity.

In some instances, expression of the recombinase, and/or the transcription factor(s) and/or differentiation factor(s) described herein may also be regulated, as described herein. For example, the differentiatable cell may comprise an exogenous nucleic acid comprising a nucleic acid sequence that encodes for the recombinase, and/or the transcription factor(s) and/or differentiation factor(s). Upon exposure to a chemical inducer or light at a particular wavelength or wavelength range, the recombinase and/or the transcription factor(s) and/or differentiation factor(s) may be expressed in the differentiatable cell.

Any appropriate system for inducing expression of a gene may be used to induce expression of a gene of interest herein (e.g., a transcription factor, differentiation factor). Examples of chemically inducible systems include, without limitations, Tet-inducible systems, cumate-inducible systems, acetaldehyde inducible systems, vanillic acid inducible systems and their derivatives, rapamycin inducible systems and derivatives thereof, and plant hormone signaling systems and derivatives thereof. Transcription of a gene of interest (e.g., gene encoding a transcription factor or differentiation factor) may be induced by the presence or absence of a chemical inducer. The chemical inducer may include, without limitation, tetracycline or a derivative thereof, cumate or a derivative thereof, acetaldehyde, vanillic acid or a derivative thereof, rapamycin or a derivative thereof, abscisic acid, gibberellin, or auxin. The chemical inducer may be a food safe additive.

In some instances, the chemical inducer regulates transcription of a gene of interest (e.g., a gene encoding a transcription factor or differentiation factor) by affecting the binding of a protein to a DNA motif regulating transcription. In some instances, the chemical inducer regulates transcription of a gene of interest (e.g., a gene encoding a transcription factor or differentiation factor) by affecting dimerization of two proteins to bring together DNA-binding and transcription regulating domains.

A Tet inducible system is derived from the E. coli tetracycline-resistance operon and uses the antibiotic tetracycline or derivatives like doxycycline as an inducer. Expression of a gene of interest (e.g., transcription factor, differentiation factor) may be inducible by tetracycline or a derivative thereof. Derivatives of tetracycline include, without limitations, doxycycline, minocycline, metacycline, and tigecycline.

Addition of tetracycline or a derivative thereof may result in activation of transcription via the Tet repressor protein (TetR). TetR binds as a homodimer to Tet operator (TetO) DNA motifs in the operon's promoter, repressing transcription of the operon. The conformation of the TetR dimer changes when bound to tetracycline, preventing it from binding to TetO elements and releasing the operon from transcriptional repression.

In some embodiments, the Tet system may be modified to function in mammalian cells. In another form, a Tet-Off system is used to regulate the transcription of a gene of interest (e.g., gene encoding a transcription factor and/or differentiation factor). In this variant, the coding sequence of protein of interest (e.g., transcription factor, differentiation factor) is placed downstream of a synthetic promoter consisting of multiple TetO elements upstream of a minimal promoter (e.g. derived from the CMV promoter) with a TATA box to initiate transcription but no enhancer elements. The cells constitutively express a fusion protein of TetR and a transcriptional activator such as the herpes simplex virus VP16 activation domain. In the absence of tetracycline, the TetR-activator fusion protein sits tightly bound to the TetO elements in the inducible promoter, activating transcription of the gene of interest. When tetracycline is present, the TetR fusion can no longer bind to the TetO elements and transcription from the promoter ceases.

In another form, a Tet-On system is used to regulate the transcription of a gene of interest (e.g., a gene encoding a transcription factor or differentiation factor). In this variant, the VP16 activation domain is fused to a mutant of TetR with reversed tetracycline-dependent behavior (reversed TetR, rTetR). In the presence of tetracycline, rTetR binds to TetO, and in its absence it does not. Therefore expression from a promoter like the one above is switched on when tetracycline is added. The rTetR or variant thereof may comprise rTetR, a high performance V16 rTetR sequence, a wildtype TetR fused to transcriptional repressor such as KRAB domain.

In some cases, transcription of a gene of interest (e.g., a gene encoding a transcription factor or a differentiation factor) is induced by the presence of cumate or a derivative thereof. In one configuration, the addition of cumate or a derivative thereof induces expression of the gene of interest. In the absence of cumate the CymR repressor protein binds to one or more CuO operator sequences placed between a strong constitutive promoter and the gene of interest, repressing transcription of the gene of interest. The addition of cumate or a derivative thereof may activate expression of the gene of interest (e.g., gene encoding a transcription factor or a differentiation factor) by interacting with at least one CymR repressor protein to prevent it from binding to CuO, thus relieving the transcriptional repression. In another configuration, transcription of the gene of interest (e.g., gene encoding a transcription factor or differentiation factor) is induced by the absence of cumate or a derivative thereof. In this example, a chimeric CymR protein fused to a transcriptional activator domain binds to CuO operator sequences upstream of a minimal promoter followed by the gene of interest in the absence of cumate, inducing expression. When cumate or the derivative thereof is supplied to the cell, it interacts with the chimeric CymR fusion protein to prevent its binding to CuO, causing the gene of interest to no longer be expressed. Further embodiments of the cumate transcriptional induction system are possible, such as those using a mutant “reverse” CymR protein that binds to CuO in the presence, rather than the absence, of cumate.

In some cases, transcription of the gene of interest (e.g., gene encoding a transcription factor or differentiation factor) is induced by the presence of acetaldehyde. In this example, Acetaldehyde-inducible regulation (AIR), a repressor from the fungus Aspergillus nidulans that binds to operator elements is placed between a constitutive promoter and the transcriptional start site, repressing transcription. In the presence of acetaldehyde, the repressor binds to acetaldehyde and, resulting in transcription of the gene of interest (e.g., gene encoding a transcription factor or differentiation factor).

In some cases, transcription of the gene of interest (e.g., gene encoding a transcription factor or differentiation factor) is induced by the presence or absence of vanillic acid. VanR protein binds to VanO sequence, and dissociates in the presence of vanillic acid. In some cases, a VAC-ON version is made by fusing VanR to a transcriptional repressor such as KRAB, or a VAC-OFF version is made by fusing it to an activator like VP16.

In some cases, transcription of the gene of interest (e.g., gene encoding a transcription factor or differentiation factor) is induced by the presence of rapamycin or a derivative thereof. Rapamycin causes the dimerization of FKBP and FRB, bringing together transcriptional activation and DNA binding domains fused to those proteins and resulting in activation of transcription.

In some cases, transcription of the gene of interest (e.g., gene encoding a transcription factor or differentiation factor) is induced by a plant hormone signaling system. The chemical inducer may comprise Abscisic acid (ABA), gibberellin (GA), or a derivative thereof. ABA triggers the dimerization of ABI and PYL1 to induce transcription of the gene of interest (e.g., gene encoding a transcription factor or differentiation factor). In some instances, a DNA binding domain is fused to AB1 or a derivative thereof and a transcriptional activator domain or derivative thereof is fused to PYL1. In some instances, GA dimerizes GID1 and GAI to induce transcription of the gene of interest (e.g., gene encoding a transcription factor or differentiation factor). In some instances, a DNA binding domain is fused to GID1 or a derivative thereof and a transcriptional activator domain or derivative thereof is fused to GAL. The DNA binding domain and the transcriptional activating domain may comprise, respectively, GAL4 and VP16; dCas9 and VPR chimeric activator; or any other combination of DNA binding domains and transcriptional activating domains.

In some cases, the chemical inducer is auxin. In such scenarios, a DNA-binding domain fused to a transcriptional repressor such as a KRAB domain and tagged with the degron is constitutively expressed and prevents expression from a promoter containing the cognate sequence for the DBD. TIR1 is also constitutively expressed. Upon the addition of auxin, the repressor fusion protein is degraded, releasing the gene of interest from transcriptional repression.

In other aspects, transcription is controlled using the light activatable system regulated by CcasS/R or UirS/R. These systems may require the addition of phycocyanobilin as a cofactor. In another instance, transcription is controlled by the CarH/CarO system, which is regulated by green light. This system may require the addition of B12 as a cofactor.

In some embodiments, the constitutively active promoters are used to drive the expression of the recombinase or to switch on the transcription factor expression after recombinase is activated. In some cases, the constitutive promoters are viral promoters. In some cases, the promoters are eukaryotic promoters. In a non-limiting example, the promoter may be cytomegalovirus immediate-early promoter (CMV promoter), SV40-early promoter (SV-40), elongation factor 1 alpha (EF1a), ubiquitin c (UBC), phosphoglycerate kinase (PGK), or beta actin (ACTB). In some cases, the recombinases can be expressed under the control of an inducible promoter (e.g., a chemically-inducible promoter, a light-inducible promoter).

In some cases, the gene of interest is one or more transcription factor or differentiation factor as described herein.

In another aspect of the methods described herein, at least one of the cells in the population of cells can be differentiated to a desired cell lineage by exposing the cell to certain conditions that allow for differentiation into the desired cell lineage. In one embodiment, at least one cell can be differentiated to a desired cell lineage by contacting the cell with a culture media that contains one or more media factors (e.g., at an appropriate concentration) that effect differentiation into the desired cell lineage. In some embodiments, at least one cell can be differentiated to a desired cell lineage by withholding or withdrawing at least one media factor from a culture media such that the cell differentiates into the desired cell lineage. In some embodiments, at least one cell can be differentiated to a desired cell lineage by cell-cell contact-mediated differentiation.

In some embodiments, different media formulations comprising at least one media factor can promote differentiation of differentiatable cells into a specific cell type or cell lineage. In some embodiments, the media components may include dexamethasone, 3-isobutyl-1-methyl xanthine (IBMX), insulin and indomethacin, triiodothyronine (t3), Asc-2-P and basic FGF (bFGF-2), transferrin, T3, cortisol, pioglitazone, ascorbic acid, calcium pantotenate, biotin, rosiglitazone, or combination thereof. In some embodiments, the media components may fetal calf serum (FCS), VEGF, FGF, platelet-derived growth factor (PDGF), epidermal growth factor (EGF), or combinations thereof. In some embodiments, the media component may include FCS, transforming growth factor-beta 1 (TGF-β1), hepatocyte growth factor (HGF), platelet-derived growth factor (PDGF), ascorbic acid, bFGF, bone morphogeneticprotein-4 (BMP-4), copper sulfate, proline, glycine, ascorbic acid, alanine, heparin, angiotensin II, sphingosylphophorylcholine, or combinations thereof.

In some embodiments, the culture media is supplemented with various media factors at a concentration of at least about 1 μM, at least about 5 μM, at least about 10 μM, at least about 15 μM, at least about 20 μM, at least about 25 μM, at least about 30 μM, at least about 35 μM, at least about 40 μM, at least about 45 μM, at least about 50 μM, at least about 60 μM, at least about 70 μM, at least about 80 μM, at least about 90 μM, at least about 100 μM, at least about 110 μM, at least about 120 μM, at least about 130 μM, at least about 140 μM, at least about 150 μM, at least about 160 μM, at least about 170 μM, at least about 180 μM, at least about 190 μM, at least about 200 μM, at least about 300 μM, at least about 400 μM, at least about 500 μM, at least about 600 μM, at least about 700 μM, at least about 800 μM, at least about 900 μM, at least about 1 mM, at least about 5 mM, at least about 10 mM, at least about 15 mM, at least about 20 mM, at least about 25 mM, at least about 30 mM, at least about 35 mM, at least about 40 mM, at least about 50 mM, at least about 60 mM, at least about 70 mM, at least about 80 mM, at least about 90 mM, or at least about 100 mM.

In some embodiments, the media formulation includes non-serum media formulations. In some embodiments, various media formulations from conventional media may be modified to enable proliferation, differentiation, or other desired qualities. In some embodiments, a media formulation comprises a synthetic media or a modification thereof. Examples of synthetic media include Minimum Essential Media (MEM), Essential 8 Media, Basal Medium Eagle (BME), Ham's F12, Ham's F-10, Fischer's Medium, CMRL-1066 Medium, Click's Medium, Medium 199, Dulbecco's Modified Eagle's Media (DMEM), RPMI-1640, L-15 medium, McCoy's 5A Modified Medium, William's Medium E, and Iscove's Modified Dulbecco's Medium (IMDM).

The differentiatable cells used in the methods provided herein may be any desired differentiatable cell. In some instances, the differentiatable cells are stem cells. In some instances, the stem cells are totipotent stem cells. In some instances, the stem cells are pluripotent stem cells. In some instances, the stem cells are multipotent stem cells. In some instances, the stem cells are oligopotent stem cells. In some instances, the stem cells are unipotent stem cells. In some cases, the stem cells are embryonic stem cells. In some cases, the stem cells are mesenchymal stem cells. In some cases, the stem cells are satellite cells or muscle stem cells. In some cases, the stem cells are fat stem cells. In some instances, the stem cells are endothelial stem cells. In some instances, the stem cells are induced pluripotent stem cells (iPSCs). In some cases, the differentiatable cells are progenitor cells. In some cases, the differentiatable cells are transit-amplifying cells (TACs). In some cases, the differentiatable cells are cells that are capable of proliferating and dividing (e.g., for a period of time, e.g., under certain conditions). In some cases, the differentiatable cells are not stem cells. For example, the differentiatable cells may be mature, somatic cells that are capable of transdifferentiation into another cell type that is different from the original cell type (e.g., under proper conditions). In some instances, the differentiatable cells are fibroblasts. In some instances, the differentiatable cells are chondroblasts. In some instances, the differentiatable cells are epithelial cells. In some instances, the differentiatable cells are erythroid-megakaryocytic cells. In some instances, the differentiatable cells are beta-cell progenitors. In some instances, the differentiatable cells are hepatic cells (e.g., hepatocytes). In some instances, the differentiatable cells are exocrine cells. In some instances, the differentiatable cells are nonsensory cells. In some instances, the differentiatable cells are non-cardiogenic mesoderm. In some instances, the differentiatable cells are cardiac fibroblasts. In some instances, the differentiatable cells are dermal fibroblasts. In some instances, the differentiatable cells are cardiomyocytes. In some instances, the differentiatable cells are fibro-adipogenic progenitor cells.

In certain embodiments, the differentiatable cells described herein are mammalian cells. In some cases, the mammalian cells are selected from the group consisting of: human cells, bovine (cow) cells, ovine (sheep) cells, porcine (pig) cells, and mouse cells. In some cases, the differentiatable cells are avian cells, such as, but not limited to, chicken cells. In some cases, the cells are fish cells, such as, but not limited to, tuna cells or salmon cells.

In another aspect of the methods disclosed herein, the population of differentiatable cells are deposited on a solid support. The solid support may allow for growth or patterning of the differentiatable cells in two dimensions. The solid support may allow for growth or patterning of the differentiatable cells in three dimensions. The solid support may be biodegradable. The solid support may comprise a natural material. Natural materials include, without limitations, extracellular matrix components, silk, gelatin, and alginate. The solid support may comprise a synthetic material. The solid support may comprise any surface or scaffold to which differentiatable cells can attach (e.g., hydrogel).

The solid support may be coated with one or more extracellular matrix components or portions or fragments thereof. For instance, the solid support may be coated with or incorporated with collagen, hyaluronic acid, fibrin, fibronectin, integrins, laminin, proteoglycans, glycosaminoglycans, gelatin, vitronectin, or any other extracellular matrix protein, or portions or fragments thereof.

The population of differentiatable cells may be deposited on a solid support in at least one layer. The population of differentiatable cells may be deposited on a solid support in at least one, two, three, four, five, six, seven, eight, nine, ten, or more layers.

The recombinases and transcription factors and/or differentiation factors described herein can be encoded by a nucleic acid. In some embodiments, a nucleic acid comprising the recombinases and transcription factors and/or differentiation factors can be an expression cassette or can be comprised within an expression cassette. As used herein, “expression cassette” means a recombinant nucleic acid construct comprising one or more nucleic acids described herein, wherein the recombinant nucleic acid construct is operably associated with at least one control sequence (e.g., a promoter).

In certain embodiments, the nucleic acid is a component of a vector that can be used to transfer the nucleic acid into a cell. As used herein, the term “vector” refers to a nucleic acid molecule capable of transporting another nucleic acid to which it has been linked. One type of vector is a genomic integration vector, or “integration vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors”. Suitable vectors comprise plasmids, bacterial artificial chromosomes, yeast artificial chromosomes, viral vectors and the like.

In the vectors, regulatory elements such as promoters, enhancers, and polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

In some aspects, the nucleic acids that are introduced into a eukaryotic cell are operably linked to a promoter and/or to a polyA signal as known in the art. In some embodiments, the nucleic acids having a 5′ end and a 3′ end is operably linked at the 5′ end to a promoter and at the 3′ end to a polyA signal. In some aspects, the nucleic acids comprise 2A peptide sequences and/or internal ribosomal entry sites.

In some embodiments, the expression cassette includes a nucleotide sequence encoding a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or whether the marker is simply a trait that one can identify through observation or testing, such as by screening (e.g., fluorescence).

### Systems

Provided herein are systems for co-differentiating a population of differentiatable cells. The systems may comprise a population of differentiatable cells, wherein each differentiatable cell of the population of differentiatable cells is engineered to contain an exogenous nucleic acid comprising: a nucleic acid sequence encoding for at least one first transcription factor or differentiation factor that effects differentiation into a first cell lineage; and a nucleic acid sequence encoding for at least one second transcription factor or differentiation factor that effects differentiation into a second cell lineage. The systems may further comprise a light configured to illuminate a first differentiatable cell of the population with light at a first wavelength or wavelength range to differentiate the first differentiatable cell into a first cell lineage.

In some instances, the systems described herein involve co-differentiation of a population of differentiatable cells into multiple cell lineages. In some instances, the systems involve co-differentiation of a population of differentiatable cells into at least two, three, four, or five cell lineages. In some embodiments, the first cell lineage and the second cell lineage are different. In some systems, at least the first cell lineage, second cell lineage, third cell lineage, fourth cell lineage, and/or fifth cell lineage are different.

In some instances, the differentiatable cells described herein comprise at least one exogenous nucleic acid. The exogenous nucleic acid may comprise a sequence encoding a transcription factor and/or differentiation factor. The sequence encoding the transcription factor and/or differentiation factor may be operably linked to a promoter. The nucleic acids described herein may comprise promoter sequences. The promoter sequences may be constitutively active. In an alternate embodiment, a promoter sequence may be conditionally active. For instance, a promoter sequence may be regulated by a chemical inducer or by light.

In the systems described herein, the differentiatable cells comprise at least one nucleic acid sequence comprising a sequence encoding at least one transcription factor and/or differentiation factor. In some embodiments, the at least one transcription factor is selected from the group consisting of: PPARγ, CEBP alpha, MYOD, MYOG, MYF5, MRF4 (MYF6), HEYL, KLF4, PAX3, SOX9, SOX5, SOX6, PRDM16, SREBP1, and any combination thereof. In certain aspects, the systems described herein may involve at least 1, 2, 3, 4, 5, 6, 7, 8, 9, 10 or more than 10 transcription factors and/or differentiation factors. Expression of the transcription factor may be regulated by a chemical inducer or by light.

In some embodiments, the differentiatable cells described herein comprise at least two exogenous nucleic acids. In some embodiments, the differentiatable cells described herein comprises at least three, four, or five exogenous nucleic acids. Each exogenous nucleic acid may comprise at least one sequence encoding a transcription factor and/or differentiation factor. The sequence encoding the transcription factor may be operably linked to a promoter.

The systems described herein comprise activatable recombinases. The activatable recombinases may comprise an activator system as described in Table 1.

In some instances, the differentiatable cell population may comprise at least two, at least three, at least four, or at least five exogenous nucleic acid sequences encoding at least two, at least three, at least four, or at least five transcription factors and/or differentiation factors.

The differentiatable cells may further comprise at least one recombinase. The differentiatable cells may further comprise one, two, three, four, five or more recombinases. At least one recombinase may be chemically-activatable as described herein. At least one recombinase may be activatable by light as described herein. In another embodiment, at least two recombinases are activatable by light.

In various aspects, a combination of light-activatable domains (e.g., a first light-activatable domain and a second light-activatable domain) may be used. In this scenario, the first light-activatable domain and the second light-activatable domain are binding partners, such that upon illumination with light at a particular wavelength or within a particular spectral range, the first and second light-activatable domains heterodimerize or hetero-oligomerize. The first and second light-activatable domains, upon illumination with light at a particular wavelength or within a particular spectral range, heterodimerize or hetero-oligomerize, thereby bringing the protein domains (or functional domains or functional portions thereof) into close contact with one another such that the recombinase(s) is/are activated. In some instances, the first and second light-activatable domains may be dissociated (e.g., thereby deactivating the recombinase) by illuminating the cell with light at a wavelength or wavelength range that is different from the wavelength or wavelength range used to heterodimerize or hetero-oligomerize the first and second light-activatable domains.

In various aspects, the light activatable domain comprises a Light-Oxygen-Voltage (LOV) photoreceptor domain, a LOV2 photoreceptor domain, a Cryptochrome (CRY) domain, Blue-light-using FAD (BLUF) photoreceptor domain, a Phytochrome (PHY) domain, CIB1 (or a functional portion of domain thereof; e.g., CIBN), a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof.

In some instances, a combination of light-activatable domains is used, wherein the first light-activatable domain is cryptochrome 2 (or a variant or a functional portion thereof) and the second-light activatable domain is CIB1 (or a variant or a functional portion or functional domain thereof; e.g., CIBN). In some instances, a combination of light-activatable domains is used, wherein the first light-activatable domain is BphP1 (or a variant or a functional portion thereof) and the second-light activatable domain is QPAS1 (or a variant or a functional portion thereof). In some cases, the light-activatable domain (or combination of light-activatable domains) is selected from Table 2. In some cases, the light-activatable domain may have an amino acid sequence having at least about 50% sequence identity (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater) to any one of the light-activatable domains described in Table 2.

In various aspects, the systems involve exposing the differentiatable cells (e.g., genetically engineered to express the fusion protein comprising the recombinase and the light-activatable domain) with light at a particular wavelength or light within a particular spectral range. The wavelength or wavelength range of light is selected such that the light is capable of activating the light-activatable domain. For example, Table 2 provides non-limiting examples of light parameters for different light-activatable domain systems. The wavelength or wavelength range of light may be one or more of infrared, near infrared, visible light (e.g., red, green, blue), ultraviolet light, or a combination thereof. Infrared light may comprise light at a wavelength or wavelength range of about 780 nm to 1 mm. Near infrared light may comprise light at a wavelength or wavelength range of about 740 nm to about 780 nm. Red light may comprise light at a wavelength or wavelength range of about 620 nm to 750 nm, 600 nm to 690 nm, or about 650 nm. Green light may comprise light at a wavelength or wavelength range of about 577 nm to about 492 nm. Blue light may comprise light at a wavelength or wavelength range of 492 to about 455 nm, or about 440 nm to about 473 nm. Ultraviolet light may comprise light at a wavelength or wavelength range from about 10 nm to 400 nm, or from about 280 to 315 nm. In various aspects, the wavelength or wavelength range of light is from 100 nm to 1 mm.

In some instances of the systems described herein, the recombinase may be configured to be expressed in an inactive form. The recombinase may comprise a chemically-induced or light-induced system in an inactive form. The recombinase may mediate irreversible excision or invert a nucleic acid sequence. The recombinase may be a serine integrase, such as #C31, TP901, and Bxb1. The recombinase may be a tyrosine recombinase, such as Cre, VCre and Flp.

In one embodiment, the recombinase is split into a first part and a second part. Addition of a chemical activator results in dimerization and activation of the recombinase. The chemical activator may be any of the chemical activators described herein, including without limitations rapamycin or derivatives thereof, ABA, GA, tetracycline or derivatives thereof, cumate or derivates thereof, or vanillic acid or derivatives thereof. The recombinase may be any recombinase, including, without limitations, Cre, VCre, Flp, #C31, TP901, and Bxb1.

In another embodiment, the first part of the recombinase and the second part of the recombinase may be fused to a light activatable domain. In various aspects, the light activatable domain comprises a Light-Oxygen-Voltage (LOV) photoreceptor domain, a LOV2 photoreceptor domain, a Cryptochrome (CRY) domain, Blue-light-using FAD (BLUF) photoreceptor domain, a Phytochrome (PHY) domain, CIB1 (or a functional portion or domain thereof; e.g., CIB1), a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof. Regulation of the dimerization and activation of the recombinase may utilize any of the light activatable systems described in Table 2. Dimerization and activation may alternatively be induced by temperature.

In another embodiment, the recombinase comprises a single chain polypeptide. The single chain polypeptide may be fused to a light activatable domain to create a light-activatable recombinase. Exposure to light at a particular wavelength or wavelength range may result in activity of the recombinase. For instance, illumination of the AsLOV2-based Cre system with blue light would result in activation of the recombinase.

In another embodiment, the recombinase may be fused to a PhoCl protein or a derivative thereof. Illumination with violet light (about 400 nm) results in cleavage of the PhoCl. In some instances, a PhoCl domain may be present in the fusion protein between a blocker domain and the recombinase domain, as depicted in FIG. 2. The blocker domain may be any domain that prevents the recombinase from functioning, such as a domain that prevents the recombinase from entering the nucleus. For instance, the blocker domain may be a steroid receptor domain which interacts with Hsp90 to prevent nuclear entry of the recombinase. Exposing the fusion protein comprising the blocker domain, the PhoCl domain, and the recombinase to violet light may result in cleavage of the PhoCl domain, allowing the recombinase to enter the nucleus, resulting in recombinase activity.

In various aspects, the systems comprise a light source configured to expose the differentiatable cells (e.g., genetically engineered to express the fusion protein comprising the recombinase and the light-activatable domain) with light at a particular wavelength or light within a particular spectral range. The wavelength or wavelength range of light is selected such that the light is capable of activating the light-activatable domain. For example, Table 2 provides non-limiting examples of light parameters for different light-activatable domain systems. The wavelength or wavelength range of light may be one or more of infrared, near infrared, visible light (e.g., red, green, blue), ultraviolet light, or a combination thereof. Infrared light may comprise light at a wavelength or wavelength range of about 780 nm to 1 mm. Near infrared light may comprise light at a wavelength or wavelength range of about 740 nm to about 780 nm. Red light may comprise light at a wavelength or wavelength range of about 620 nm to 750 nm, 600 nm to 690 nm, or about 650 nm. Green light may comprise light at a wavelength or wavelength range of about 577 nm to about 492 nm. Blue light may comprise light at a wavelength or wavelength range of 492 to about 455 nm, or about 440 nm to about 473 nm. Ultraviolet light may comprise light at a wavelength or wavelength range from about 10 nm to 400 nm, or from about 280 to 315 nm. In various aspects, the wavelength or wavelength range of light is from 100 nm to 1 mm.

In various aspects, the systems involve illuminating the cells with light having one or more illumination parameters. In some cases, the one or more illumination parameters includes light intensity and/or a temporal pattern of illumination. In some cases, the illumination intensities can be about 0 μW/mm² to about 100 μW/mm². In some cases, the illumination intensities can be at least or up to about 0 μW/mm², 0.1 μW/mm², 0.2 μW/mm², 0.3 μW/mm², 0.4 μW/mm², 0.5 μW/mm², 0.6 μW/mm², 0.7 μW/mm², 0.8 μW/mm², 0.9 μW/mm², 1 W/mm², 1.2 μW/mm², 1.4 W/mm², 1.6 μW/mm², 1.8 μW/mm², about 2 μW/mm², about 3 μW/mm², about 4 μW/mm², about 5 μW/mm², about 6 μW/mm², about 8 μW/mm², about 10 μW/mm², about 20 μW/mm², about 30 μW/mm², about 40 μW/mm², about 50 μW/mm², about 60 μW/mm², about 70 μW/mm², about 80 μW/mm², about 90 μW/mm², or about 100 μW/mm². In some cases, the temporal pattern may include a stimulus duration and an interstimulus duration. In some cases, the temporal pattern comprises a light stimulus duration of at least about one tenth of a second, at least about 1 second, at least about 1 minute, at least about 5 minutes, at least about 30 minutes, or at least about 1 hour. In some cases, the stimulus duration may be at least about 5 minutes. In some cases, the temporal pattern comprises an interstimulus duration of at least about 1 second, at least about 1 minute, at least about 5 minutes, at least about 10 minutes, at least about 30 minutes, at least about 1 hour, at least about 2 hours, at least about 3 hours, at least about 4 hours, at least about 5 hours, or greater. In some cases, the interstimulus duration may be from about 20 minutes to about 250 minutes.

In various aspects, the systems and methods provided herein may be used for various applications. In some embodiments, the systems and methods provided herein may be used to produce cultured or cultivated meat. For example, the systems and methods provided herein may be used to produce a cultured or cultivated meat composition comprising two or more different cell types that are integrated into a single, continuous tissue, and in a spatially-patterned manner such that the type of cell in different regions of the composition can be controlled or biased toward one cell type or another. In other aspects, the systems and methods provided herein may be used to create tissues (e.g., tissue engineering). For example, the systems and methods provided herein may be used to create tissues comprising multiple different cell types with spatial specificity (e.g., for biomedical purposes (e.g., skin grafts, pacemaker), for research and development (R&D), for in vitro tissue experimentation including reproducing growth of organoids for drug testing or disease modeling).

In some embodiments, the one or more light comprises one or more light-emitting diodes (LEDs). In some cases, the one or more LEDs comprises at least two different LEDs. In some cases, the at least two different LEDs emit light at different wavelengths or wavelength ranges. Additionally or alternatively, the one or more light source comprises one or more lasers. Additionally or alternatively, the one or more light source comprises an incandescent light source.

The differentiatable cells used in the systems provided herein may be any desired differentiatable cell. In some instances, the differentiatable cells are stem cells. In some instances, the stem cells are totipotent stem cells. In some instances, the stem cells are pluripotent stem cells. In some instances, the stem cells are multipotent stem cells. In some instances, the stem cells are oligopotent stem cells. In some instances, the stem cells are unipotent stem cells. In some cases, the stem cells are embryonic stem cells. In some cases, the stem cells are mesenchymal stem cells. In some cases, the stem cells are satellite cells or muscle stem cells. In some cases, the stem cells are fat stem cells. In some instances, the stem cells are endothelial stem cells. In some instances, the stem cells are induced pluripotent stem cells (iPSCs). In some cases, the differentiatable cells are progenitor cells. In some cases, the differentiatable cells are transit-amplifying cells (TACs). In some cases, the differentiatable cells are cells that are capable of proliferating and dividing (e.g., for a period of time, e.g., under certain conditions). In some cases, the differentiatable cells are not stem cells. For example, the differentiatable cells may be mature, somatic cells that are capable of transdifferentiation into another cell type that is different from the original cell type (e.g., under proper conditions). In some instances, the differentiatable cells are fibroblasts. In some instances, the differentiatable cells are chondroblasts. In some instances, the differentiatable cells are epithelial cells. In some instances, the differentiatable cells are erythroid-megakaryocytic cells. In some instances, the differentiatable cells are beta-cell progenitors. In some instances, the differentiatable cells are hepatic cells (e.g., hepatocytes). In some instances, the differentiatable cells are exocrine cells. In some instances, the differentiatable cells are nonsensory cells. In some instances, the differentiatable cells are non-cardiogenic mesoderm. In some instances, the differentiatable cells are cardiac fibroblasts. In some instances, the differentiatable cells are dermal fibroblasts. In some instances, the differentiatable cells are cardiomyocytes. In some instances, the differentiatable cells are fibro-adipogenic progenitor cells.

In certain embodiments, the differentiatable cells described herein are mammalian cells. In some cases, the mammalian cells are selected from the group consisting of human cells, bovine (cow) cells, ovine (sheep) cells, porcine (pig) cells, and mouse cells. In some cases, the differentiatable cells are avian cells, such as, but not limited to, chicken cells. In some cases, the cells are fish cells, such as, but not limited to, tuna cells or salmon cells.

In another aspect of the systems disclosed herein, the systems further comprise a solid support configured to deposit the population of differentiatable cells are deposited. The solid support may be configured to allow for growth or patterning of the differentiatable cells in two dimensions. The solid support may be configured to allow for growth or patterning of the differentiatable cells in three dimensions. The solid support may be biodegradable. The solid support may comprise a natural material. Natural materials include, without limitations, extracellular matrix components, silk, gelatin, and alginate. The solid support may comprise a synthetic material. The solid support may comprise any surface or scaffold to which differentiatable cells can attach (e.g., hydrogel).

The solid support may be coated with one or more extracellular matrix components, or portions of fragments thereof. For instance, the solid support may be coated with collagen, hyaluronic acid, fibrin, fibronectin, integrins, laminin, proteoglycans, glycosaminoglycans, gelatin, vitronectin, or any other extracellular matrix protein, or portions or fragments thereof.

The population of differentiatable cells may be deposited on a solid support in at least one layer. The population of differentiatable cells may be deposited on a solid support in at least one, two, three, four, five, six, seven, eight, nine, ten, or more layers.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

The term “differentiatable cell” refers to any cell capable of differentiating from a first cell type to a second, different cell type under certain conditions. In some instances, a differentiatable cell is a cell that can divide and renew itself over a period of time (e.g., under certain conditions). In some instances, the differentiatable cell includes stem cells, such as, but not limited to totipotent stem cells, pluripotent stem cells, multipotent stem cells, oligopotent stem cells, unipotent stem cells, or induced pluripotent stem cells (iPSCs). In some cases, the differentiatable cells include progenitor cells. In some cases, the differentiatable cells include transit-amplifying cells (TACs). In some cases, the term differentiatable cells includes cell that are capable of transiently or permanently proliferating and dividing. In some cases, the term differentiatable cell includes a mature somatic cell capable of transdifferentiating into a cell type different from the original cell type under certain conditions.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993).

The section headings used herein are for organizational purposes only and are not to be construed as limiting the subject matter described.

### EXAMPLES

### Example 1: Recombinase-Mediated Excision to Activate Fate Cassette Expression

A population of differentiatable cells containing the exogenous nucleic acid sequence is cultured for codifferentiation. Two pairs of recognition sites, each pair recognized by a different recombinase, are placed in the construct such that, when the corresponding recombinase is active in the cell, either one fate determining cassette or the other is permanently switched on. In this scheme, one recognition sequence for each recombinase is placed between the leftmost constitutive promoter and the transcriptional start site for the gene controlled by that promoter. The two fate cassettes are placed at the opposite end of the construct; each is preceded by a recognition sequence for one of the recombinases and a transcriptional start site, but no promoter. In this state the fate cassettes should not be expressed, as there is no promoter directly upstream to recruit the transcriptional machinery due to the presence of the blocking sequence. When a recombinase is active in the cell, all of the sequence between its two recognition sites is excised, bringing the fate cassette immediately downstream of its rightmost recognition site under control of the leftmost constitutive promoter. In this case, if recombinase 2 is activated, the entire sequence between the promoter and the muscle cassette is excised, the muscle cassette is expressed, and the adipose cassette remains in the genome but is silent as the muscle cassette intervenes between it and the promoter. If recombinase 1 is activated (whether recombinase 2 is active or not), all the sequence between its recognition sites is excised, including the muscle cassette, and the adipose cassette is expressed.

A transcriptional inducer is added which induces expression of recombinase 1 and recombinase 2 in all cells. Both recombinases are expressed but are inactive. Expression of recombinase 2 is activated in all cells by the addition of a chemical inducer. The entire sequence between the recognition sites of recombinase 2 is activated. Muscle cell fate is permanently activated in all cells, while expression of adipose fate is silent.

A subset of cells are illuminated with light to activate recombinase 2. The muscle cell transcription factor is excised in the cells that are illuminated and adipose fate is permanently activated.

### Example 2: Co-Differentiation of a Population of Differentiatable Cells to Either Fat or Muscle with Light and a Chemical Inducer

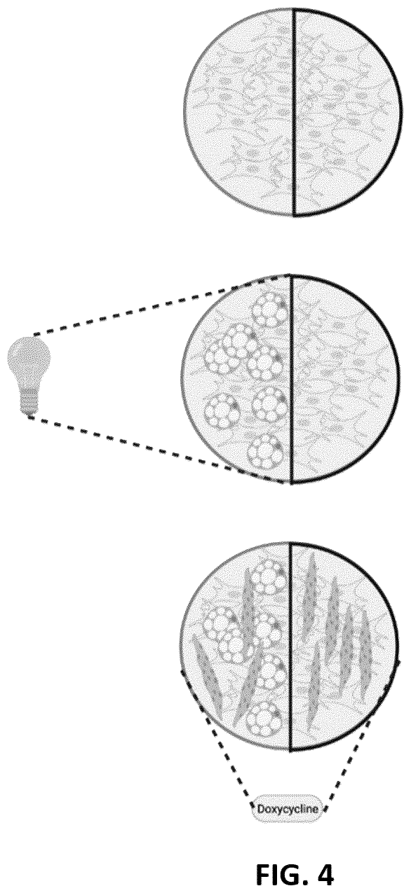

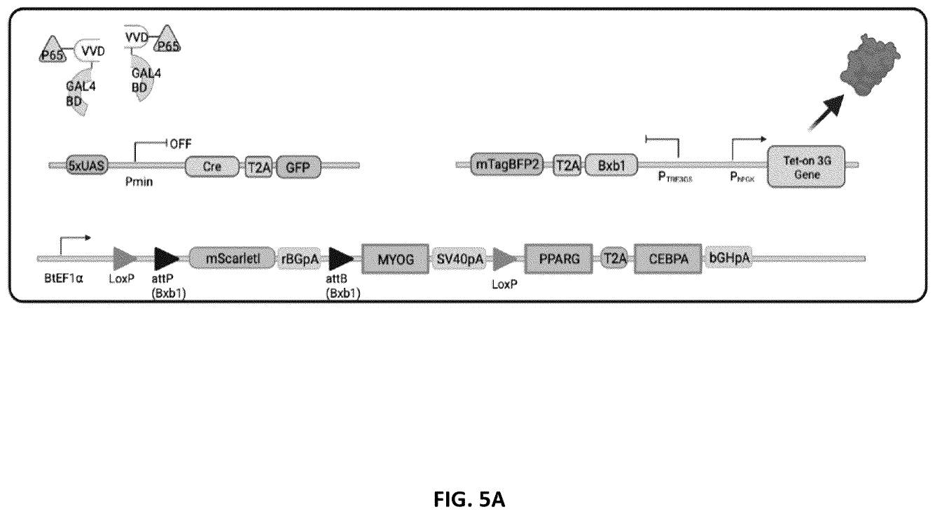

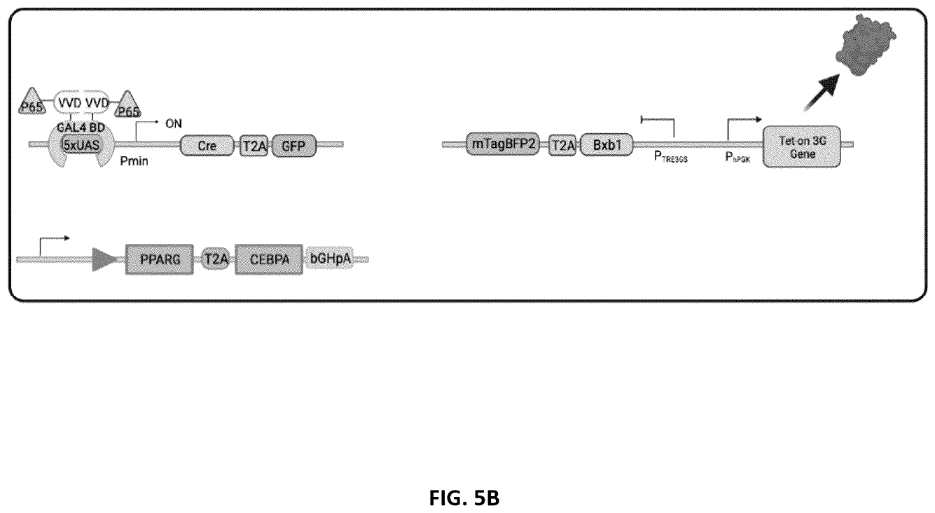

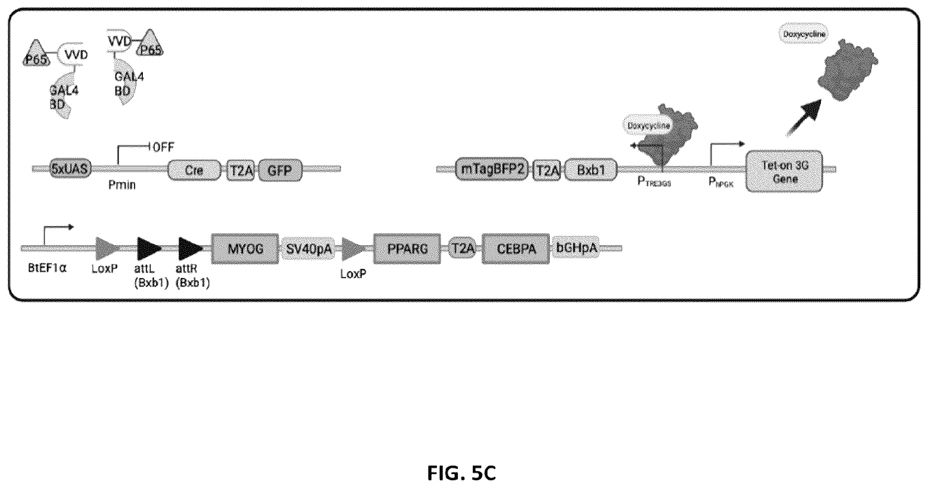

FIG. 4 is a schematic providing an overview of a process used to co-differentiate a population of differentiatable cells (fibroblasts in this example) to fat and muscle, as described in this example. FIGS. 5A-5C depicts a non-limiting example of the co-differentiation mechanism and constructs, as described in this example. Briefly, a photomask was used so that only a portion of the cells in the well receive light. All cells were engineered to express a light-responsive promoter driving Cre recombinase expression, a doxycycline-responsive promoter driving Bxb1 recombinase expression and a DNA construct containing myogenic and adipogenic transcription factors, along with Cre and Bxb1 recognition sites. Initially a portion of the cells on the left of the well were illuminated. This lead to Cre recombinase and GFP expression. The Cre recombinase recognizes LoxP sites and recombines the DNA such that MYOG is excised and the adipogenic transcription factors PPARG and CEBPA are expressed, leading to fibroblast transdifferentiation towards fat. After 2 days, doxycycline was added to the media, leading to Bxb1 expression in cells. Bxb1 recognizes attP/attB target sites in cells not recombined by light-induced Cre expression, resulting in excision of the DNA between these target sites and generating attL/attR sites in their place. Through this process, MYOG expression occurs in cells which were not recombined by light-induced Cre expression. Expression of MYOG enabled fibroblast transdifferentiation to myotubes. Since both light-responsive Cre expression and recombination are usually not 100% efficient, MYOG expression and therefore myotube formation was expected to occur on both the dark and illuminated part of the well.

Table 3 describes the plasmids and lentiviral vectors used in this example.

TABLE 3
Plasmids and lentiviral vectors used in this example.

| Name | Type | Construct Details |
|---|---|---|
| P_733 | Plasmid | P_{UBC}-PhyA-Gal4DBD-bGHpA |
| P_734 | Plasmid | P_{UBC}-FHY1-VP64-bGHpA |
| P_735 | Plasmid | P_{UBC}-Gal4DBD-COP1-bGHpA |
| P_736 | Plasmid | P_{UBC}-p65-UVR8-bGHpA |
| P_738 | Plasmid | P_{UBC}-Gal4DBD-VVD-p65-bGHpA |
| P_740 | Plasmid | Gal4UAS₅-P_{MLPmin}-Cre-T2A-EGFP-bGHpA |
| P_745 | Plasmid | P_{Ef1α}-loxP-attP-mScarletI-rBGpA-attB-MyoG- |
| | | SV40pA-loxP-PPARG-T2A-CEBPA-bGHpA |
| P_854 | Plasmid for | P_{TRE3GS}-Bxb1-T2A-mTagBFP2-SV40pA-P_{hPGK}- |
| | lentiviral | TetOn3G-P_{SV40}-PuroR-WPRE |
| | vector | |

### I. Engineering SV40 Fibroblast to Express Cre Recombinase and GFP in Response to Light (P_740)

Stable integration of the light-inducible gene expression system was achieved by Sleeping Beauty transposon. 500,000 SV40 fibroblasts (fibroblasts stably expressing SV40 large T antigen) were resuspended in 100 μL P1 Primary Cell Nucleofector Solution with Supplement 1 (Lonza, V4XP-1024) containing 4.5 μg of plasmid DNA (P_738: P_740 for the ratio 10:1, 5:1, 2:1 and 1:1; P_733: P_734: P_740 or P_735: P_736: P_740 for the ratio 3:1:1, 5:5:1, 2:2:1 and 1:1:1) and 0.5 μg Sleeping Beauty transposase SB100X mRNA (Vector Builder, R009S). Nucleofection was performed in Nucleocuvette using program CA-137 of Lonza 4D-Nucleofector® X Unit. After incubation for 30 minutes at room temperature, cells were transferred into growth medium for culture.

Cells transfected with the same light-inducible gene expression system were pooled together 48 hours post-transfection and expanded in a T75 flask. The light-responsive cells were always incubated in the dark or handled under green (UV and red light-inducible system) or red (blue light-inducible system) light to minimize the induction of GFP expression by ambient light. Cells transfected with P_738 were exposed to 2.5 μW/mm² of blue light (465 nm) with a 20 seconds ON, 60 seconds OFF pulsing pattern. Cells transfected with P_733 and P_734 were exposed to 1.5 μW/mm² of red light (630 nm) with a 402 seconds ON, 3198 seconds OFF pulsing pattern after addition of 10 μM phycocyanobilin (SiChem, SC-1800). Cells transfected with P_735 and P_736 were exposed to 0.15 μW/mm² of 310 nm UV light, with 1 minute ON, 29 minutes OFF pulsing pattern. After 2 days of illumination, cells were detached and dissociated into single cell suspension using TrypLE. After centrifugation, cells were resuspended in PBS and filtered through a 70 μm cell strainer. Cell sorting of GFP low and high populations was performed using SONY cell sorter SH800S. All the sorted cell populations were expanded in the growth medium in the dark. Sorting of GFP negative population was performed after expansion for 7 days in the dark in order to remove the cells with residual Cre-GFP expression. To assess light-inducible gene expression, a portion of the cells was cultured in the dark or under light for 2 days, and GFP expression was measured by flow cytometry. Approximately 30% UV-responsive cells, 35% blue-responsive cells and 8% red-responsive cells expressed GFP, indicating that not all sorted cells responded to light stimulation.

### II. Engineering SV40Fibroblasts to Express Bxb1 Recombinase (P_854)

Lentiviral vectors were generated by adding plasmid P_854 to 1,000,000 Lenti-X 293T cells (Takara, 632180) together with a Lenti-X packaging single shot (Takara).

50,000 cells containing either: A) P_738, P_740 (blue light responsive promoter); or B) P_735, P_736, P_740 (UV light responsive promoter) were seeded to each well in a 12 well plate in growth medium the day before transduction. On the day of transduction, growth medium was replaced before adding P_854 lentiviral vectors at MOI 10 and polybrene at 8 μg/ml for transduction. Cells were incubated with lentiviral vectors for 24 hours before doxycycline (Sigma, D3072-1 ML) treatment at 2 μg/ml. Cell sorting (Sony SH800S) of BFP positive populations was performed 48 hours post doxycycline treatment to isolate the transduced populations. Sorted cell populations were expanded in the growth medium without doxycycline in the dark.

### III. Engineering Cells to Express Transcription Factors in Response to Light and Chemical Stimulus (P_745)

100,000 cells containing (i) P_738, P_740, and P_854, or (ii) P_735, P_736, P_740, and P_854 were seeded to each well in a 6 well plate in growth medium the day before transfection. On the day of transfection, 2.5 μg of P_745 were co-transfected with 0.25 μg of PiggyBac hyPB plasmid (Vector Builder, VB900088-2874gzt) using 10 μL of Lipofectamine Stem Transfection Reagent (ThermoFisher, STEM00015) per well according to the manufacturer's instructions. Medium was replaced 48 hours after transfection. Following expansion in the dark, cells were detached and dissociated into single cell suspension using TrypLE. After centrifugation, cells were resuspended in PBS and filtered through a 70 μm cell strainer. Cell sorting of mScarlet-positive populations was performed using SONY cell sorter SH800S.

### IV. Light-Mediated Spatial and Temporal Control of Gene Expression

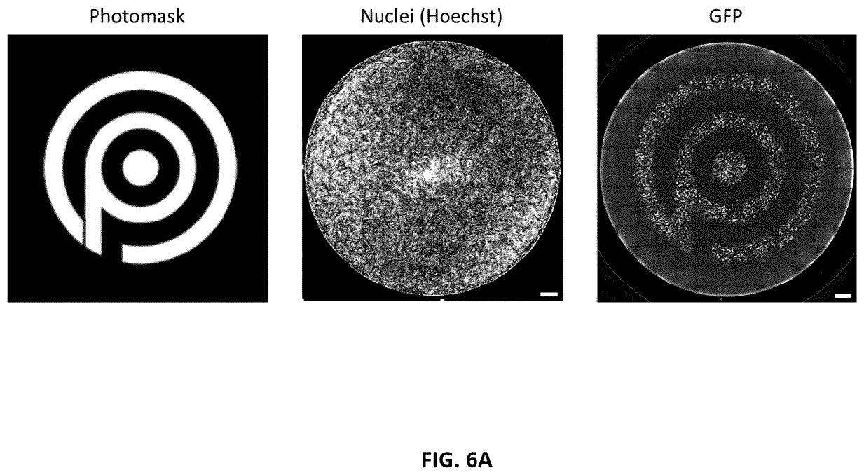

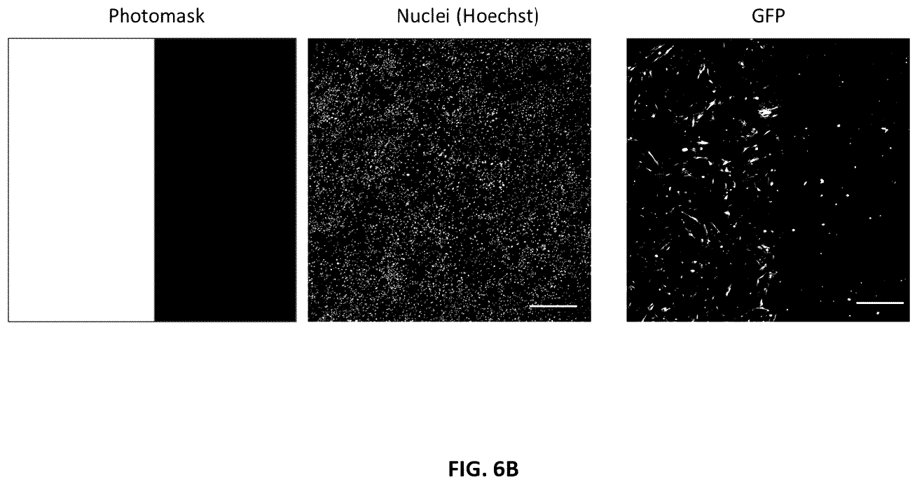

SV40 fibroblasts stably engineered with P_740 and P_738 were plated in growth medium. The following day, cells were spatially illuminated using a photomask with blue light (465 nm) of 2.5 μW/mm² with a pulsing pattern of 20 seconds ON, 60 seconds OFF. After 48 hours of illumination, the cells were spiked with 5 μg/ml Hoechst 33342 and live imaged on a Leica DMi8 thunder microscope using a 20×0.8NA objective. As shown in FIGS. 6A and 6B, scale bar: 500 μm only the cells which were exposed to light expressed the fluorescent protein, demonstrating spatial and temporal control of gene expression (e.g., cell differentiation pattern) using the methods and systems described herein.

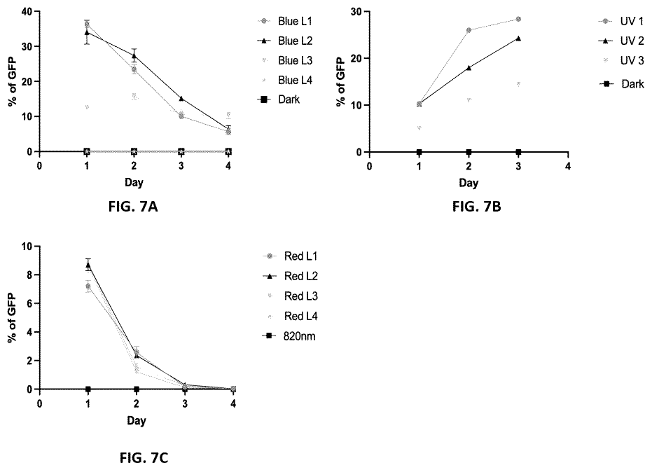

To further demonstrate light-mediated control of gene expression, various light parameters were tested. 3800 cells comprising (i) P_738 and P_740, or (ii) P_733, P_734, and P_740, or (iii) P_735 P_736, and P_740 were seeded to each well in a 96 well plate in growth medium the day before illumination. Medium supplemented with 10 μM phycocyanobilin (SiChem, SC-1800) was added to the wells with P_733, P_734 and P_740 cells. Cells transfected with P_738, P_740 were either exposed to dark conditions or blue light (465 nm) of 5 (L1), 2.5 (L2), 1 (L3) or 0.5 (L4) μW/mm² with a 20 seconds ON, 60 seconds OFF pulsing pattern. Cells transfected with P_733, P_734, and P_740 were either exposed to 10 μW/mm² of infrared (820 nm) constant illumination in order to keep the light responsive promoter inactive, or red light (630 nm) of 4 (L1), 2 (L2), 1 (L3) or 0.5 (L4) μW/mm² with a 1 minute ON, 29 minutes OFF pulsing pattern. Cells transfected with P_735, P_736, and P_740 were either exposed to dark conditions or UV light of 0.15 (UV 1), 0.09 (UV 2) or 0.04 (UV 3) μW/mm² with a 1 minute ON, 29 minutes OFF pulsing pattern. As shown in FIGS. 7A-7C, the ratio of GFP-positive cells was assessed by flow cytometry. The results demonstrated dynamic GFP expression across multiple light systems (e.g., blue light, UV light, and red light). For the blue responsive promoter, L2 was chosen for subsequent experiments. For the UV responsive system, UV1 was chosen for subsequent experiments.

### V. Co-Differentiation Using Light and Doxycycline

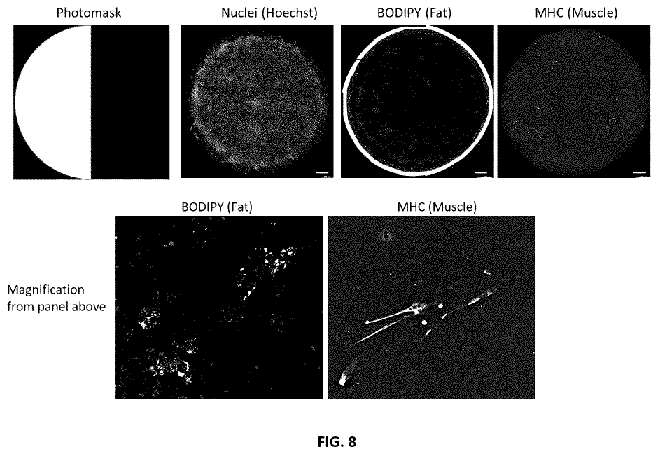

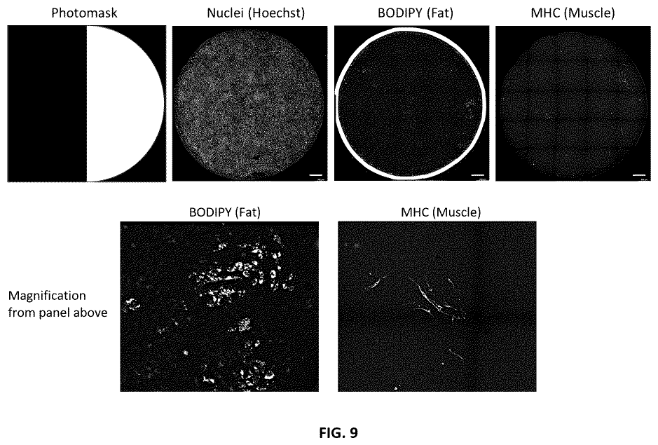

A 96 well plate was coated with 100 μg/ml collagen overnight, washed with PBS and dried. Cells containing either A) P_738 P_740 P_854 P_745 (blue light-responsive system, FIG. 8), or B) P_735 P_736 P_740 P_854 P_745 (UV light-responsive system, FIG. 9) were seeded in growth medium at 4000 cells per well of a 96 well plate. The following day, the medium was changed to co-differentiation medium, and the cells were illuminated with blue (465 nm) of 2.5 μW/mm² with a pulsing pattern of 20 seconds ON, 60 seconds OFF, or 0.15 μW/mm² of 310 nm UV light, with 1 minute ON, 29 minutes OFF pulsing pattern for 2 days. The following day, half the co-differentiation medium was exchanged, and doxycycline was added to a final concentration of 2 μg/ml. 3 days later, half the co-differentiation medium was changed. 4 days later, cells were fixed with 4% PFA before being permeabilized with PBS containing 0.2% Triton-X100 and then blocked with PBS containing 2% BSA and 0.1% Tween-20. Cells were stained with mouse anti-myosin heavy chain antibody (MHC) (DSHB, A4.1025-c; 1:100) at 4° C. overnight. The following day, cells were stained with donkey anti-mouse AF647 antibody (Thermo Fisher Scientific, A32787; 1:2000), BODIPY (Cayman Chemical Company, 25892), and DAPI (Abcam, ab228549; 10 μM) at room temperature for 1 hour before washing and imaging. Images were taken on a Leica DMi8 thunder microscope using a 10×0.3NA objective. FIG. 8 demonstrates that fibroblasts illuminated with blue light preferentially differentiated to fat. Cells which were not recombined by light induced Cre-recombinase expression remained doxycycline-responsive and differentiated towards muscle. FIG. 9 demonstrates that fibroblasts illuminated with UV light preferentially differentiated to fat. Cells which were not recombined by light induced Cre-recombinase expression remained doxycycline-responsive and differentiated towards muscle.

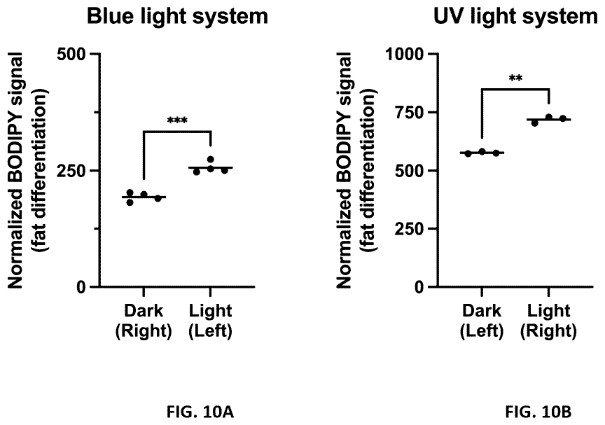

FIGS. 10A and 10B depict quantitative measurement of lipid accumulation in cells in FIG. 8 and FIG. 9. FIGS. 10A and 10B demonstrate that fibroblasts illuminated with blue light (FIG. 10A) or UV light (FIG. 10B) preferentially differentiate to fat cells. Scale bar: 500 μm. Lower images are magnifications of areas of the BODIPY and MHC wells above.

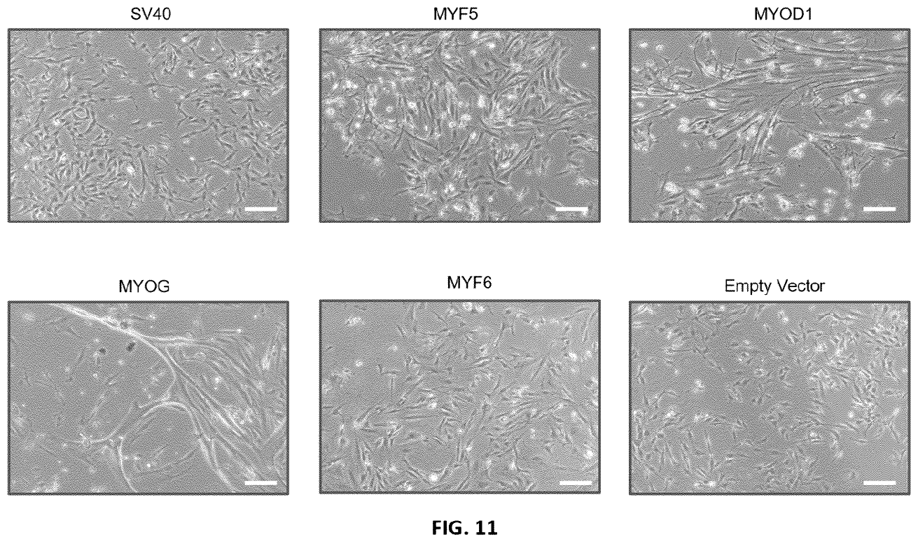

FIG. 11 depicts constitutive expression of different transcription factors or differentiation factors in fibroblasts. SV40 fibroblasts were transduced with one of the following lentiviral vectors: P_{CMV}-MYF5, P_{CMV}-MYOD1, P_{CMV}-MYOG, P_{CMV}-MYF6, or empty vector. Cells were incubated with the lentiviral vectors for 48 hours before selection with puromycin. Transduced cells were cultured in growth medium without puromycin after selection. 5 days post transduction, images were taken. Cells expressing MYOD1 and MYOG exhibited distinct morphological change and did not replicate further. MYF5 and MYF6 also exhibited morphological changes, although it was not as distinct as MYOD1 and MYOG. Hence, this indicates that various transcription/differentiation factors could be expressed in fibroblasts which caused the fibroblasts to transdifferentiate towards myotubes. Scale bar: 100 μm.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-78. (canceled)

79. A method of co-differentiating a population of differentiatable cells, the method comprising:
(a) providing or obtaining the population of differentiatable cells;
(b) controlling differentiation of a first differentiatable cell of the population of differentiatable cells with light, thereby differentiating the first differentiatable cell into a first cell lineage; and
(c) controlling differentiation of a second differentiatable cell of the population of differentiatable cells with a non-light-based inducer, thereby differentiating the second differentiable cell into a second cell lineage,
wherein the first cell lineage and the second cell lineage are different, thereby co-differentiating the population of differentiatable cells.

80. The method of claim 79, wherein the controlling differentiation of (b) comprises illuminating the first differentiatable cell with light at a first wavelength or wavelength range or wherein the controlling differentiation of (b) comprises removing light at a first wavelength or wavelength range from the first differentiatable cell.

81. The method of claim 79, wherein the controlling differentiation of (c) comprises contacting the second differentiatable cell of the population of differentiatable cells with a chemical inducer to differentiate the second differentiatable cell into the second cell lineage; or the controlling differentiation of (c) comprises removing a chemical inducer from the second differentiatable cell of the population of differentiatable cells to differentiate the second differentiatable cell into the second cell lineage.

82. The method of claim 79, wherein each differentiatable cell of the population of differentiatable cells is engineered to contain an exogenous nucleic acid comprising:
(i) a nucleic acid sequence encoding for at least one first transcription factor or first differentiation factor that effects differentiation into the first cell lineage; and
(ii) a nucleic acid sequence encoding for at least one second transcription factor or second differentiation factor that effects differentiation into the second cell lineage.

83. The method of claim 82, wherein the controlling differentiation of (b) comprises modulating expression of the at least one first transcription factor or first differentiation factor, and the controlling differentiation of (c) comprises modulating expression of the at least one second transcription factor or second differentiation factor.

84. The method of claim 83, wherein the exogenous nucleic acid comprises at least one promoter operably linked to the nucleic acid sequence encoding the at least one first transcription factor or first differentiation factor and nucleic acid sequence encoding the at least one second transcription factor or second differentiation factor.

85. The method of claim 84, wherein the at least one promoter is a constitutive promoter.

86. The method of claim 85, wherein the exogenous nucleic acid further comprises:
(iii) a blocking sequence downstream of the at least one promoter which, when present, blocks expression of the at least one first transcription factor or first differentiation factor and/or the at least one second transcription factor or second differentiation factor.

87. The method of claim 85, wherein the nucleic acid sequence encoding the at least one first transcription factor or first differentiation factor, or a portion thereof, is present in the exogenous nucleic acid in reverse orientation such that the at least one first transcription factor or first differentiation factor is not expressed.

88. The method of claim 85, wherein the nucleic acid sequence encoding the at least one second transcription factor or second differentiation factor, or a portion thereof, is present in the exogenous nucleic acid in reverse orientation such that the at least one second transcription factor or second differentiation factor is not expressed.

89. The method of claim 83, wherein each differentiatable cell of the population of differentiatable cells further comprises:
(iv) an exogenous nucleic acid sequence encoding a first activatable recombinase; and
(v) an exogenous nucleic acid sequence encoding a second activatable recombinase.

90. The method of claim 89, wherein the first activatable recombinase is a light-activatable recombinase.

91. The method of claim 89, wherein the second activatable recombinase is a light-activatable recombinase.

92. The method of claim 89, wherein the first activatable recombinase and the second activatable recombinase are different.

93. The method of claim 89, wherein the second activatable recombinase is a chemically-activatable recombinase.

94. The method of claim 89, wherein expression of the first activatable recombinase, expression of the second activatable recombinase, or both, is induced by a chemical inducer, or is induced by light.

95. The method of claim 94, wherein the blocking sequence is flanked by a first recombinase recognition site that is recognized by the first activatable recombinase, a second recombinase recognition site that is recognized by the second activatable recombinase, or both.

96. The method of claim 95, wherein the controlling differentiation of (b), the controlling differentiation of (c), or both, results in excision of the blocking sequence thereby inducing expression of the at least one first transcription factor or first differentiation factor, the at least one second transcription factor or second differentiation factor, or both.

97. The method of claim 79, wherein the population of differentiatable cells comprises stem cells, multipotent stem cells, mature somatic cells capable of transdifferentiation under certain conditions, or human differentiatable cells or bovine differentiatable cells.

98. The method of claim 79, wherein the first cell lineage, and/or the second cell lineage are selected from the group consisting of: an adipocyte, a myocyte, and a chondrocyte.

99. The method of claim 82, wherein the at least one first transcription factor, the at least one second transcription factor, or both, is selected from the group consisting of: PPARγ, CEBP alpha, MYOD, MYOG, MYF5, MRF4 (MYF6), HEYL, KLF4, PAX3, PRDM16, SREBP1, SOX9, SOX5, SOX6, and any combination thereof.

100. The method of claim 79, wherein the controlling differentiation of (b) and the controlling differentiation of (c) occur substantially simultaneously or sequentially.

101. The method of claim 100, wherein the controlling differentiation of (b) precedes the controlling differentiation of (c), or the controlling differentiation of (c) precedes the controlling differentiation of (b).

102. A system for co-differentiating a population of differentiatable cells, the system comprising:
(a) the population of differentiatable cells, wherein each differentiatable cell of the population of differentiatable cells is engineered to contain an exogenous nucleic acid comprising:
(i) a nucleic acid sequence encoding for at least one first transcription factor or first differentiation factor that effects differentiation into a first cell lineage; and
(ii) a nucleic acid sequence encoding for at least one second transcription factor or second differentiation factor that effects differentiation into a second cell lineage; and
(b) one or more light source configured to control differentiation of a first differentiatable cell of the population of differentiatable cells with light at a first wavelength or wavelength range to differentiate the first differentiatable cell into the first cell lineage,
wherein the first cell lineage and the second cell lineage are different.
